(19) United States
(12) Patent Application Publication
(10) Pub. No.: US 2007/0244314 A1
(43) Pub. Date: Oct. 18, 2007

(54) Method For Extracting Nucleic Acid And Nucleic Acid-Extracting Apparatus

(75) Inventor: Toshihiro Mori, Asaka-shi (JP)

Correspondence Address: BIRCH STEWART KOLASCH & BIRCH, PO BOX 747, FALLS CHURCH, VA, 22040-0747, US

(73) Assignee: FUJIFILM CORPORATION, Tokyo (JP)

(21) Appl. No.: 11/596,666

(22) PCT Filed: May 17, 2005
(86) PCT No.: PCT/JP05/09308

§ 371 Date: Nov. 16, 2006

(30) Foreign Application Priority Data
May 18, 2004 (JP) 2004-148365
Jun. 29, 2004 (JP) 2004-190954

(51) Int. Cl. C07H 21/00; B01L 11/00

(52) U.S. Cl. 536/25.41; 422/101

(57) ABSTRACT

A method for extracting nucleic acid comprising: adsorbing nucleic acid to a nucleic acid-adsorptive solid carrier by contacting a sample solution containing nucleic acid with the nucleic acid-adsorptive solid carrier; washing the nucleic acid-adsorptive solid carrier by contacting a washing solution with the nucleic acid-adsorptive solid carrier, while the nucleic acid is adsorbed to the nucleic acid-adsorptive solid carrier; and desorbing the nucleic acid from the nucleic acid-adsorptive solid carrier by contacting a recovering solution with the nucleic acid-adsorptive solid carrier, wherein at least one of a time where the washing solution is dispensed, and allowed to stand; and a time where the recovering solution is dispensed and allowed to stand is controlled to a predetermined time.

### TECHNICAL FIELD

The present invention relates to a method for extracting nucleic acid where nucleic acid in a sample solution is automatically extracted using a nucleic acid-extracting cartridge equipped with a filter material and also to an apparatus for extracting nucleic acid.

### BACKGROUND ART

As to the related method for extracting nucleic acid, there are methods where centrifugal force is applied, magnetic beads are used, a filter is used, etc. For example, there is a method where nucleic acid is adsorbed with a solid phase such as silicon dioxide, silica polymer or magnesium silicate and then purified by succeeding operations such as washing and desorption (refer, for example, to Japanese Patent Publication No. 07/051,065).

### DISCLOSURE OF THE INVENTION

However, although the above-mentioned related methods for extracting nucleic acid are good in terms of separating property, they are not sufficient in terms of simplicity, quickness and automation adaptability. There are also problems that an industrial large-scale production of an adsorptive medium having same properties is difficult, that handling is inconvenient and that molding into various shapes is difficult, etc.

The present invention has been achieved in view of the above-mentioned circumstances and its object is to provide a method for extracting nucleic acid where, in a method of separating and purifying nucleic acid by adsorption of nucleic acid in a sample solution containing nucleic acid with a nucleic acid-adsorptive solid carrier followed by subjecting to desorption via washing, etc., it is possible to prepare a sample solution containing nucleic acid in good efficiency, simplicity, quickness, good automation adaptability and good reproducibility and also to provide an apparatus for extracting nucleic acid.

The above-mentioned object of the present invention is able to be achieved by the following constitutions.

(1) A method for extracting nucleic acid comprising:

adsorbing nucleic acid to a nucleic acid-adsorptive solid carrier by contacting a sample solution containing nucleic acid with the nucleic acid-adsorptive solid carrier;

washing the nucleic acid-adsorptive solid carrier by contacting a washing solution with the nucleic acid-adsorptive solid carrier, while the nucleic acid is adsorbed to the nucleic acid-adsorptive solid carrier; and

desorbing the nucleic acid from the nucleic acid-adsorptive solid carrier by contacting a recovering solution with the nucleic acid-adsorptive solid carrier,

wherein at least one of a time where the washing solution is dispensed and allowed to stand; and a time where the recovering solution is dispensed and allowed to stand is controlled to a predetermined time.

(2) The method as described in (1) above,

wherein the time where the washing solution is allowed to stand is controlled to a range of 50 seconds to 1,000 seconds.

(3) The method as described in (1) above,

wherein the time where the washing solution is allowed to stand is controlled to a range of 100 seconds to 300 seconds.

(4) The method as described in any of (1) to (3) above,

wherein the time where the recovering solution is allowed to stand is controlled to a range of 20 seconds to 300 seconds.

(5) The method as described in any of (1) to (3) above,

wherein the time where the recovering solution is allowed to stand is controlled to a range of 25 seconds to 60 seconds.

(6) The method as described in any of (1) to (5) above,

wherein the nucleic acid-adsorptive solid carrier is a nucleic acid-adsorptive solid carrier that adsorbs nucleic acid by an interaction involving substantially no ionic bond.

(7) The method as described in any of (1) to (6),

wherein a method for preparing the sample solution containing nucleic acid comprises:

mixing a pretreating solution containing a compound selected from a chaotropic salt, a surfactant, an antifoaming agent, a proteinase and a nucleic acid stabilizer with a test sample, so as to obtain a mixed solution; and

adding a water-soluble organic solvent to the mixed solution.

(8) The method as described in (7) above,

wherein the nucleic acid stabilizer is a mercapto compound.

(9) The method as described in (7) or (8) above,

wherein the chaotropic salt is a guanidium salt.

(10) The method as described in (7) to (9) above,

wherein the water-soluble organic solvent comprises at least one of methanol, ethanol, propanol and butanol.

(11) The method as described in any of (1) to (10) above,

wherein the nucleic acid-adsorptive solid carrier is received in an inner part of a container having at least two openings of a nucleic acid-extracting cartridge, and

wherein the method comprises:

adsorbing the nucleic acid in the sample solution to the nucleic acid-adsorptive solid carrier by a pressure difference after dispensing the sample solution containing the nucleic acid to the nucleic acid-extracting cartridge;

removing impurities by a pressure difference after dispensing the washing solution to the nucleic acid-extracting cartridge; and

separating the nucleic acid adsorbed to the nucleic acid-adsorptive solid carrier from the nucleic acid-adsorptive solid carrier by a pressure difference after dispensing the recovering solution to the nucleic acid-extracting cartridge, so as to recover the nucleic acid along with the recovering solution.

(12) A nucleic acid-extracting apparatus for conducting a method as described in (11) above,

wherein a nucleic acid-adsorptive solid carrier is a filter material.

(13) The nucleic acid-extracting apparatus as described in (12) above,

wherein the nucleic acid-extracting apparatus automatically carries out an extracting operation by a pressurization, and

wherein the extracting operation comprises:

adsorbing a nucleic acid in a sample solution to the filter material after dispensing the sample solution containing the nucleic acid to a nucleic acid-extracting cartridge;

removing impurities after dispensing a washing solution to the nucleic acid-extracting cartridge; and

separating the nucleic acid adsorbed to the filter material from the filter material after dispensing a recovering solution to the nucleic acid-extracting cartridge, so as to recover the nucleic acid along with the recovering solution.

(14) The nucleic acid-extracting apparatus as described in (12) or (13) above, comprising:

a compressed air supplying mechanism that introduces a compressed air from a pressurizing nozzle to the nucleic acid-extracting cartridge; and

a dispensing mechanism comprising a first dispensing nozzle that dispenses a washing solution to the nucleic acid-extracting cartridge and a second dispensing nozzle that dispenses a recovering solution to the nucleic acid-extracting cartridge.

(15) The nucleic acid-extracting apparatus as described in any of (12) to (14) above, comprising:

a retaining mechanism that retains a plurality of arranged nucleic acid-extracting cartridges and a plurality of arranged recovering containers for receiving the recovering solution containing nucleic acid;

a compressed air supplying mechanism that introduces a compressed air from a single pressurizing nozzle to the plurality of arranged nucleic acid-extracting cartridges;

a dispensing mechanism comprising a first dispensing nozzle that dispenses a washing solution to the plurality of arranged nucleic acid-extracting cartridges and a second dispensing nozzle that dispenses a recovering solution to the plurality of arranged nucleic acid-extracting cartridges; and

a transfer means that relatively transfers one of the single pressurizing nozzle of the compressed air supplying mechanism and the retaining mechanism against the other.

In accordance with the constitution as such, an operation for extracting nucleic acid where the nucleic acid-extracting cartridge and recovering container receiving the recovering solution containing nucleic acid are arranged in plural each and retained by a retaining mechanism, a single nozzle of the compressed air supplying mechanism and dispensing nozzles of a dispensing mechanism are subjected to a relative transfer so that introduction of compressed air into a nucleic acid-extracting cartridge and dispensing of washing solution and recovering solution is conducted and nucleic acid adsorbed with the filter material in the nucleic acid-extracting cartridge is separated and recovered is now able to be carried out by a simple mechanism.

(16) The nucleic acid-extracting apparatus as described in any of (12) to (15) above,

wherein the plurality of arranged nucleic acid-extracting cartridges are supported at a fixed side, and

wherein the single pressurizing nozzle of the compressed air supplying mechanism is supported in movable in an arranging direction of the plurality of arranged nucleic acid-extracting cartridges.

In accordance with the constitution as such, when a pressurizing nozzle is transferred along the direction of arranging the nucleic acid-extracting cartridge, it is now possible to successively supply the air to plural nucleic acid-extracting cartridges.

(17) The nucleic acid-extracting apparatus as described in any of (12) to (16) above,

wherein at least one of the first dispensing nozzle and the second dispensing nozzle, and the single pressurizing nozzle are installed in an unified transferable matter.

In accordance with the constitution as such, when the compression nozzle and dispensing nozzle are installed in the unified transferable matter, it is now possible to simply constitute the compressed air supplying mechanism and dispensing mechanism.

(18) The nucleic acid-extracting apparatus as described in any of (12) to (17) above,

wherein the single pressurizing nozzle of the compressed air supplying mechanism is supported at a fixed side, and

wherein the plurality of arranged nucleic acid-extracting cartridges are supported in movable in an arranging direction of the plurality of arranged nucleic acid-extracting cartridges.

In accordance with the constitution as such, when the dispensing nozzle is fixed and the nucleic acid-extracting cartridge is transferred, large amount of nucleic acid-extracting cartridge is able to be subjected to each of the treatments of pressurization, dispensing, washing, dispensing and recovering successively and, when the already-treated nucleic acid-extracting cartridge and recovering container are exchanged with the untreated ones successively, treatment in large amount in a continuous manner is possible.

(19) The nucleic acid-extracting apparatus as described in any of (12) to (18) above,

wherein the plurality of nucleic acid-extracting cartridges are installed in a same arranging distance, and

wherein an arranging distance from each of the first dispensing nozzle and the second dispensing nozzle to the single pressurizing nozzle is an integral multiple of the same arranging distance of the plurality of nucleic acid-extracting cartridges.

In accordance with the constitution as such, a nucleic acid-extracting cartridges are installed in the same arranging pitch and the arranging pitch of the dispensing nozzle to the pressurizing nozzle is an integral multiple of the arranging pitch of the nucleic acid-extracting cartridge whereby it is now possible that plural different nucleic acid-extracting cartridges are subjected to a dispensing treatment and a pressurizing treatment at the same time and shortening of the treating time is achieved.

### BRIEF DESCRIPTION OF THE DRAWING

FIG. 1 is an embodiment of the nucleic acid-extracting apparatus and is an oblique view showing the state where a cover is removed;

FIG. 2 is an outline constitutional drawing of a transfer head of the nucleic acid-extracting apparatus;

FIG. 3 is an outline block constitutional drawing of the nucleic acid-extracting apparatus;

FIG. 4A is an oblique view and FIG. 4B is a cross-sectional view along IVB-IVB of the cartridge;

FIG. 5A to G are step charts for extracting operation;

FIG. 6A to C are illustrative drawings which show the mode of supplying of compressed air to the cartridge from the transfer head;

FIG. 7A to B are illustrative drawings which show the mode of dispensing of the washing solution to the cartridge from the transfer head;

FIG. 8A to B are illustrative drawings which show the mode of dispensing of the recovering solution to the cartridge from the transfer head;

FIG. 9 is an outline constitutional drawing which shows another embodiment of the nucleic acid-extracting apparatus;

FIG. 10 is a flow chart which shows the proceeding of the nucleic acid-extracting steps concerning the present invention;

FIG. 11 is a graph which shows the relation between the yield of nucleic acid and the time for being allowed to stand after infusion of the washing solution; and

FIG. 12 is a graph which shows the relation between the yield of nucleic acid and the time for being allowed to stand after infusion of the recovering solution.

2 denotes a main body of the apparatus, 3 denotes a retaining mechanism, 4 denotes a mechanism for supplying compressed air, 5 denotes a dispensing mechanism, 6 denotes a rack, 7 denotes a transfer means, 1 denotes a cartridge (nucleic acid-extracting cartridge), 1b denotes a nucleic acid-adsorptive porous membrane, 12 denotes a waste solution container, 13 denotes a recovering container, 21 denotes a cartridge holder, 22 denotes a container retaining stand, 40 denotes a transfer head (transferable body), 41 denotes a pressurizing nozzle, 43 denotes an air pump, 45 denotes an open-and-close valve, 46 denotes a pressure sensor, 51w, 51r each denotes a dispensing nozzle, 52w, 52r each denotes a supplying pump, 56w, 56r each denotes a bottle, 70 denotes a control part, 72 denotes a memory part, 100 denotes a nucleic acid-extracting apparatus, S denotes a sample solution, W denotes a washing solution, R denotes a recovering solution.

### BEST MODE FOR CARRYING OUT THE INVENTION

As hereinafter, detailed illustration will be given for the mode for carrying out the nucleic acid-extracting apparatus which is suitable for conducting the nucleic acid-extracting method of the present invention.

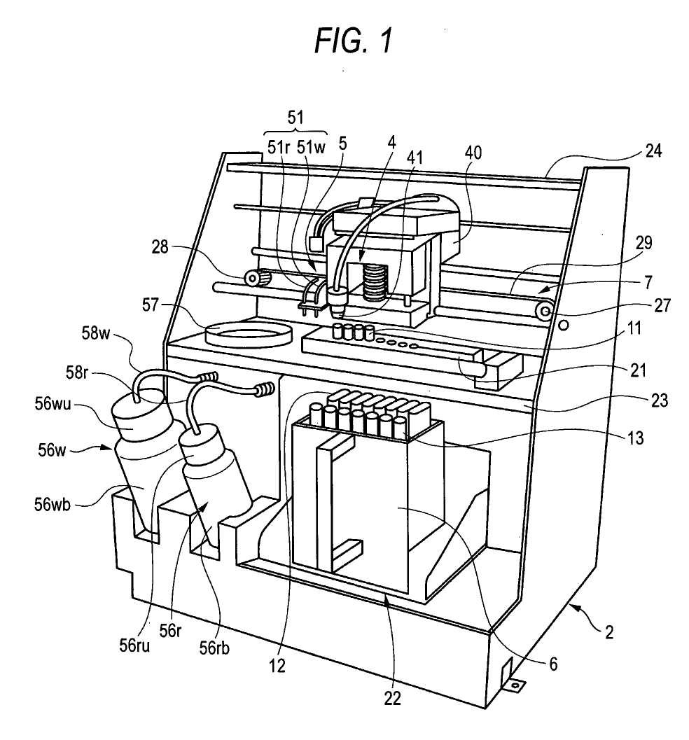

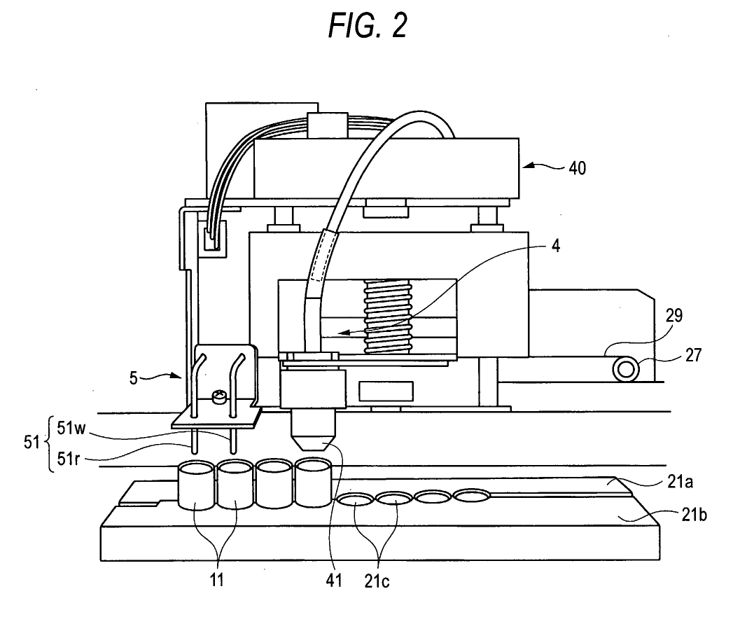

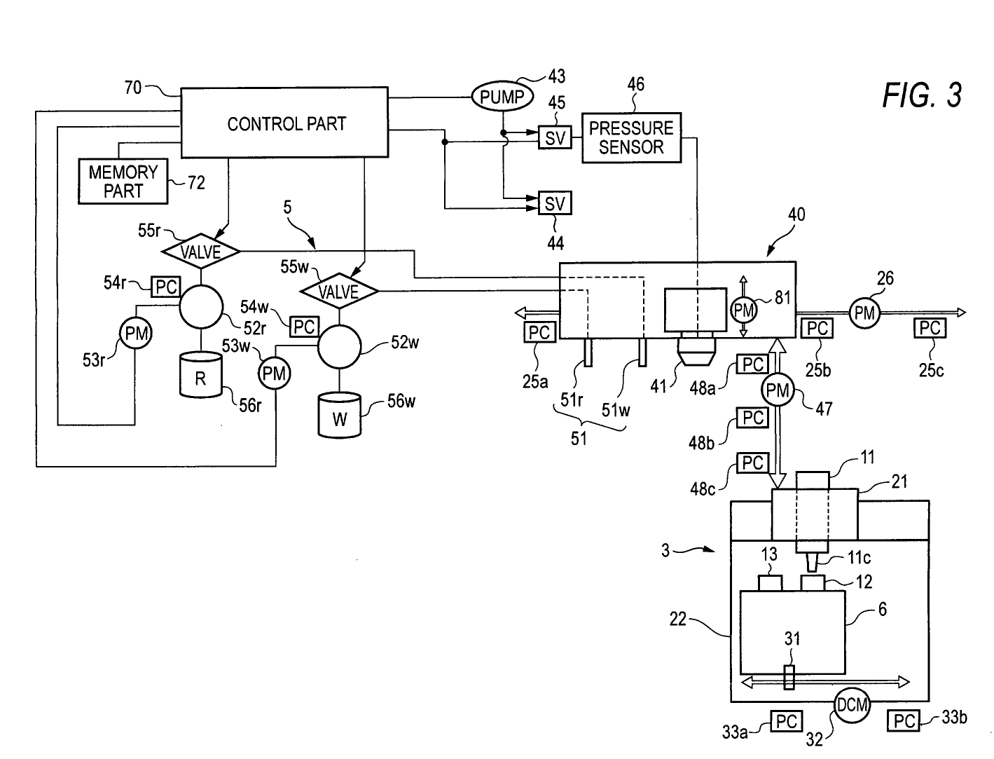

FIG. 1 is an embodiment of the nucleic acid-extracting apparatus and is an oblique view showing the state where a cover is removed; FIG. 2 is an outline constituting drawing of a transfer head of the nucleic acid-extracting apparatus; and FIG. 3 is an outline block constituting drawing of the nucleic acid-extracting apparatus.

The present nucleic acid-extracting apparatus 100 is constituted by being equipped with a retaining mechanism 3 where each plural nucleic acid-extracting cartridges (hereinafter, just referred to as “cartridges”) receiving a filter material and recovering containers 13 receiving a recovering solution containing nucleic acid are retained and arranged in a container; a compressed air supplying mechanism 4 where compressed air is introduced from a single pressurizing nozzle 41 to cartridges 1; a dispensing mechanism 5 having a dispensing nozzle 51 which dispenses each of a washing solution and a recovering solution to the cartridges 1; and a transfer mechanism 7 where the retaining mechanism 10 and pressurizing nozzle 41 of the compressed air supplying mechanism 20 are subjected to a relative transfer. With regard to the filter material, a nucleic acid-adsorptive solid carrier such as a nucleic acid-adsorptive porous substance (here, nucleic acid-adsorptive porous membrane) may be used.

In the nucleic acid-extracting apparatus 100 where the nucleic acid-extracting method according to the present invention is conducted, (1) a step where a sample solution containing nucleic acid is passed through a nucleic acid-adsorptive porous membrane so that nucleic acid is adsorbed with said porous membrane, (2) a step where said nucleic acid-adsorptive porous membrane is washed under such a state that nucleic acid is adsorbed and (3) a step where the recovering solution is passed through said nucleic acid-adsorptive porous membrane so that nucleic acid is separated from said porous membrane are successively carried out.

Before illustrating the mechanism of the nucleic acid-extracting apparatus 100 in this embodiment, a step of extracting nucleic acid by this nucleic acid-extracting apparatus will be illustrated.

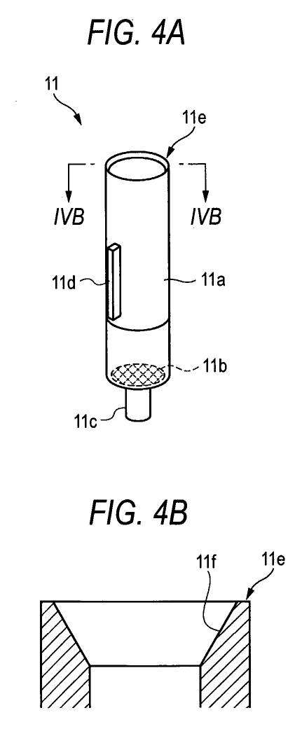

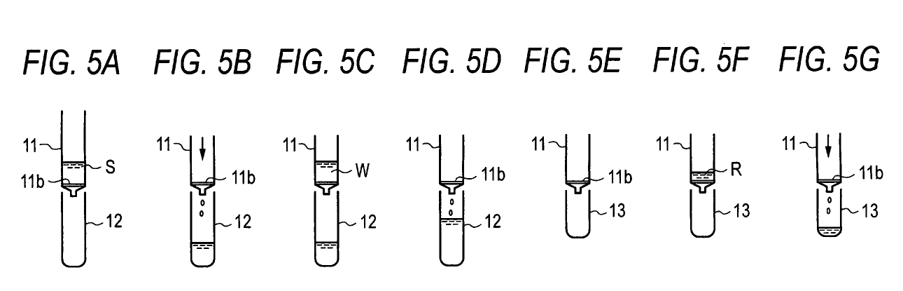

FIG. 4A is an oblique view and FIG. 4B is a cross-sectional view along IVB-IVB of the cartridge; and FIG. 5A to G comprise drawings which show steps of the extracting operation.

The nucleic acid-extracting apparatus 100 is to extract nucleic acid in a sample solution using a cartridge 11 as shown in FIG. 4A. The cartridge 11 comprises in such a manner that a nucleic acid-adsorptive porous membrane 11b is retained at the bottom of a cylindrical main body 11a where upper end is open, the area below the nucleic acid-adsorptive porous membrane 11b of the cylindrical main body 11a is formed in a funnel-like shape, an outlet 11c in a form of finely tubular nozzle is projected in a predetermined length to the center of the lower end and projection 11d in a longitudinal direction is formed on both sides of the cylindrical main body 11a. After sample solution, washing solution and recovering solution which will be mentioned later are dispensed from the upper opening 11e of the cartridge 11, compressed air is introduced from the upper opening 11e and each solution is passed through the nucleic acid-adsorptive membrane 11b, flown down and discharged from the outlet 11c to a waste liquid container 12 or a recovering container 13 which will be mentioned later. Incidentally, in the case as shown in the drawing, the cylindrical main body 11a is in such a structure that it is divided into upper part and lower part and attached by engaging them. As shown in FIG. 4B as a cross-sectional view along IVB-IVB, the upper opening 11e has a slope surface 11f where inner surface is cut in a taper-form and the slope surface 11f is formed in such a manner that it is almost identical with the outer surface of the slope of the front end of a pressurizing nozzle 41 which will be mentioned later.

Basically, the nucleic acid-extracting apparatus 100 carries out extraction of nucleic acid by extracting steps as shown in FIG. 5A to G. Firstly, in a step FIG. 5A, a sample solution S containing nucleic acid which is subjected to a dissolving treatment is poured into a cartridge 11 located on a waste liquid container 12. Then, in a step FIG. 5B, compressed air is introduced into a cartridge 11 to pressurize and passed through a nucleic acid-adsorptive porous membrane 11b so that the sample solution S is passed, nucleic acid is adsorbed with the nucleic acid-adsorptive porous membrane 11b and the liquid components which passed therethrough are discharged to a waste liquid container 12.

Then, in a step FIG. 5C, a washing solution W is automatically dispensed to the cartridge 11, compressed air is introduced into the cartridge 11 in a step FIG. 5D, washing and removal of other impurities are conducted while nucleic acid is still retained in the nucleic acid-adsorptive porous membrane 11b and the washing solution W which passed therethrough is discharged to a waste liquid container 12. The steps FIGS. 5C and D may be repeated for several times.

Then, after the waste liquid container 12 of the lower part of the cartridge 11 is exchanged to a recovering container 13 in a step FIG. 5E, the recovering solution R is automatically dispensed to the cartridge 11 in a step FIG. 5F, compressed air is introduced into the cartridge 11 in a step FIG. 5G to pressurize so that bonding force between the nucleic acid-adsorptive porous membrane 11b and nucleic acid is made weak to release the adsorbed nucleic acid and a recovering solution R containing nucleic acid is discharged to a recovering container 13 to recover.

Fundamentally, the nucleic acid-adsorptive porous membrane 11b in the above cartridge 11 is a porous substance where liquid is able to pass through and its surface has a characteristic that nucleic acid in the sample solution is adsorbed by means of a chemical bonding force. It is constituted in such a manner that, upon washing with a washing solution, its adsorption is retained while, upon recovering by a recovering solution, adsorptive force of nucleic acid is made weak so that it is released. Details will be illustrated later.

As shown in FIG. 1 to FIG. 3, the nucleic acid-adsorptive apparatus 100 is equipped, in the main body 2 of the apparatus, with a cartridge holder 21 for retaining plural cartridges 11, a container retaining stand 22 which retains a waste liquid container 12 and a recovering container 13, a compressed air supplying mechanism 4 by which compressed air is introduced into a cartridge 11, a dispensing mechanism 5 by which a washing solution W and a recovering solution R are dispensed to the cartridge 11, etc. The cartridge holder 21 and the container retaining stand 22 constitute a retaining mechanism 3. Now, each of the mechanisms 3 to 5 will be specifically illustrated.

Retaining Mechanism

A retaining mechanism 3 comprises a cartridge holder 21 and a container retaining stand 22. In the container retaining stand 22, there is a rack 6 retaining a waste liquid container 12 and a recovering container 13 below the front side of the main body of the apparatus 2. Transfer for exchanging the container of the rack 6 (forward and backward transfer) is conducted by transfer of a working material 31 (refer to FIG. 3) set at the basement of the container retaining stand 22 by driving of a container-exchanging motor 32 (DC motor). As a result of such forward and backward transfer, the recovering container 13 is positioned below the cartridge holder 21 or the waste liquid container 12 is positioned below the cartridge holder 21. Operation of the above-mentioned container-exchanging motor 32 is controlled corresponding to the detection by position sensors 33a, 33b.

A cartridge holder 21 is constituted in a two-divided structure by adhesion of front and back plate materials and is equipped with retaining materials 21a, 21b extending in a transverse direction. Plural retaining holes 21c are formed in the retaining materials 21a, 21b, a cartridge 11 is inserted from upside and the lower end of the projections 11d (refer to FIGS. 4A and B) formed on both sides of the cylindrical main body 11a of the cartridge 11 is connected to and retained by a connecting material (not shown) in the cartridge holder 21. The connecting material is able to be transferred and, upon transfer, connection to the projection 11d is released and all cartridges 11 are made to fall down and discharged at the same time.

At the position where the rack 6 is descended as shown in FIG. 1, a lower end of the outlet 11c of the cartridge 11 retained in a cartridge holder 21 is positioned at upper side than the waste solution container 12 and recovering container 13 set at the rack 6. When the container retaining stand 22 is ascended and descended by driving of an elevating motor 47 (refer to FIG. 3) such as a pulse motor and the rack 6 is ascended and descended by control accompanied by detection by photo sensors 48a to 48c, a predetermined amount of an outlet 11c of the cartridge 11 is inserted into a waste solution container 12 or a recovering container 13 when the rack 6 is elevated.

On its upper surface, the rack 6 is equipped with waste solution-retaining holes and recovering container-retaining holes extending in a transverse direction in parallel two lines and each of plural waste solution containers 12 and plural recovering containers 13 are retained at the waste solution container-retaining holes on rear side and the recovering container-retaining holes on front side, respectively, in lines. The waste solution container-retaining holes and the recovering container-retaining holes are arranged in same positions and same pitches (distances) as the retaining holes 21c of the cartridge holder 21 and setting is conducted in such a manner that each of the waste solution container 12 and the recovering container 13 is positioned below each of the retained cartridges 11. With regard to the waste solution container 12 and the recovering container 13, it is preferred to use containers having different size, shape, etc. for prevention of confusion.

Compressed Air Supplying Mechanism

As shown in FIG. 1 to FIG. 3, a compressed air supplying mechanism 4 is equipped with a transfer head 40 as a transferable substance which ascends and descends against the rack 6 of the above-mentioned retaining mechanism 3, a single pressurizing nozzle 41 set at the transfer head 40, an air pump 43 which generates compressed air, a relief valve 44, a value for opening and closing the air supplying path set at the side of the pressurizing nozzle 41, a pressure sensor 46 set at the side of a pressurizing nozzle 41 and a means for ascending and descending the nozzle which ascends and descends the pressurizing nozzle 41. With regard to a means for descending and ascending the nozzle, an operation of ascending and descending is achieved by a nozzle ascending/descending motor 81 such as a pulse motor and a bolt-nut mechanism connected thereto. As a result of the constitution as such, compressed air is successively supplied to the cartridges 11. The operation of each of air pump 43, relief valve 44 and pressurizing nozzle 41 is conducted on the basis of a control order from the control part 70.

The above-mentioned transfer head 40 is equipped with a head transfer motor 26 (refer to FIG. 3) such as a pulse motor as a transfer means located between a middle frame 23 and an upper frame 24 of the main body of the apparatus 2, a pulley 27 at a driving side which is driven and rotated by a head transfer motor 26, a pulley 28 at an idler side which is freely rotated and conducts a tension adjustment and a timing belt 29 which is hung between the pulley 27 of a driving side and the pulley 28 of an idler side. Incidentally, the head transfer motor 26 is driven by control as a result of detection by photo sensors 25a to 25c and transfers the transfer head 40 along the arranged direction of the cartridge 11.

A pressurizing nozzle 41 is installed in a transfer head 40 in an up-and-down transferable manner with much downward movement and an external surrounding of lower front end of the pressurizing nozzle 41 is made conical. As a result thereof, when the pressurizing nozzle 41 transfers downward and front end of the pressurizing nozzle 41 is made to contact to the upper end opening of the cartridge 11 set in a cartridge holder 21, a slope 1f of a cartridge which is cut in a taper form tightly adheres to the conical surface at the front end of the pressurizing nozzle 41 and the inner area of the cartridge 11 is tightly closed. Under such a tightly sealed state, it is now possible to supply the compressed air without leakage into a cartridge 11.

A relief valve 44 is made open to air and is operated when air in the path between the air pump 43 and the opening-closing valve 45 is discharged. An air circuit is constituted in such a manner that the open-close valve 45 is selectively opened and compressed air from an air pump 43 is introduced into a cartridge 11 via a pressurizing nozzle 41. As a result of the constitution as such, an air flow-supplying path is formed from the air pump 43 to the cartridge 11.

Dispensing Mechanism

A dispensing mechanism 5 is equipped with a nozzle 51w for dispensing a washing solution and a nozzle 51r for dispensing a recovering solution which are installed together in the above-mentioned transferable head 40 which is able to be transferred in a transverse direction on a cartridge holder 21, a pump 52w (refer to FIG. 3) for supplying a washing solution where a washing solution W received in a washing solution bottle 56w is supplied and sent to a nozzle 51w for dispensing a washing solution, a pump 52r (refer to FIG. 3) for supplying a recovering solution where a recovering solution R received in a recovering solution bottle 56r is supplied and sent to a nozzle 51r for dispensing a recovering solution, a waste solution container 57 installed in an intermediate frame 23, etc.

A transfer head 40 stops at each cartridge 11 by a heat transfer motor 26 (refer to FIG. 3) and, in a returning state, stops on a waste solution container 57 so that driving is controlled so as to make the space on each cartridge open. When the space on each cartridge 11 opens, working ability is greatly improved.

The nozzle 51w for dispensing a washing solution and the nozzle 51r for dispensing a recovering solution are bent downward in their front ends, the nozzle 51w for dispensing a washing solution is connected to a pump 52w for supplying a washing solution via a valve 55w, a pump 52w for supplying a washing solution is connected to a washing solution bottle 56w, a nozzle 51r for dispensing a recovering solution is connected to a pump 52r for supplying a recovering solution via a valve 55r and a pump 52r for supplying a recovering solution is connected to a recovering solution bottle 56r. Each of the washing solution bottle 56w and the recovering solution bottle 56r is attached to the front side of the main body of the apparatus 2 whereby an operation property is enhanced. The pump 52w for supplying a washing solution and the pump 52r for a recovering solution are constituted by a tube pump and each of them is controlled for driving so that a predetermined amount of the washing solution W and the recovering solution R each is dispensed on the basis of a position detection by sensors 54w and 54r by pump motors 53w and 53r (pulse motor). Those pump motors 53w, 53r and valves 55w, 55r are operated depending upon the instruction from the control part 70.

When the washing solution W or the recovering solution R is dispensed, the valve 55w or 55r is opened, the pump motor 53w or 53r is driven and rotor materials of the pump 52w for supplying a washing solution or the pump 52r for supplying a recovering solution is rotated and operated. As a result thereof, the washing solution W or the recovering solution R is sucked by the pump 52w for supplying a washing solution or the pump 52r for supplying a recovering solution and is discharged from the nozzle 51w for dispensing the washing solution or the nozzle 51r for dispensing the recovering solution via the valve 55w or 55r. Upon the discharge as such, the nozzle 51w for dispensing the washing solution or the nozzle 51r for dispensing the recovering solution is transferred on the cartridge 11. As a result, a predetermined amount of the washing solution W or the recovering solution R is dispensed in the cartridge 11.

Each of a washing solution bottle 56w and a recovering solution bottle 56r comprises container main body 56wb, 56rb and cap 56wu, 56ru; in each of both caps 56wu, 56ru, a suction pipe 58w, 58r in a fine pipe shape is installed, the lower end of said suction tube 58w, 58w is opened near the bottle of the container main body 56wb, 56rb and sucks the washing solution W and the recovering solution R corresponding to the action of the pump 52w for supplying a washing solution or the pump 52r for supplying a recovering solution.

Each of the mechanisms 3 to 5 as mentioned above is controlled by a linked control part 70 corresponding to the input operation of an operation panel (not shown) installed at the upper part of the main body of the apparatus 2. In other words, driving and control are conducted on the basis of a program previously memorized in a memory part 72 connected to the control part 70.

Now, an extracting operation by the above-mentioned nucleic acid-extracting apparatus 100 will be specifically illustrated. Firstly, a cartridge 11 is set in a cartridge holder 21 in a rack 6 of the retaining mechanism 3, each of the waste solution container 12 and the recovering container 13 is set in the rack 6 and the rack 6 is installed in a container retaining stand 22 of the main body of the apparatus 2 to prepare. Then a sample solution S which was subjected to a dissolving treatment is successively infused into each cartridge 11 using a pipette or the like. At that time, a transfer head 40 is positioned immediately on the waste solution container 57 making the space on the cartridge open. Incidentally, it is also possible that a sample solution S is previously infused into a cartridge 11 set before or after setting in the rack 6 before installing in the nucleic acid-extracting apparatus 100.

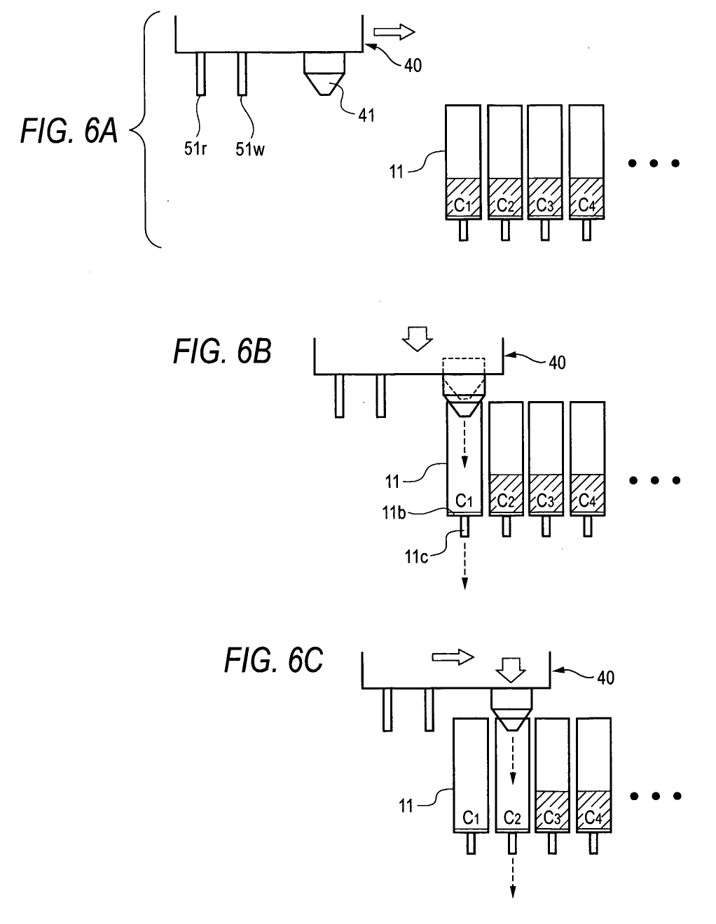

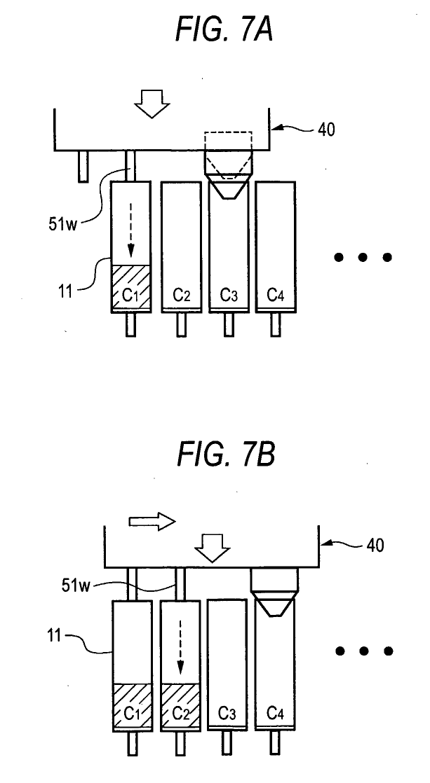

After that, the apparatus is made to act by operation of an operation panel whereupon, as shown in FIG. 6A, a transfer head 40 transfers to the position immediately on the cartridge 11. A pressurizing nozzle 41 is placed immediately on the cartridge C1 which is the left end in the drawing being shown as an example and a pressurizing nozzle 41 of the transfer head 40 is transferred downward (FIG. 6B) by driving a nozzle ascending/descending motor 81 of the compressed air supplying mechanism 4. As a result, outer surface of the front end of the pressurizing nozzle 41 closely adheres to the slant side 11f of the cartridge 11. In the meanwhile, a container retaining stand 22 moves upward by driving of an elevation motor 47, a predetermined amount of the lower end outlet 11c of the cartridge 11 is inserted into the waste solution container 12 so as to prevent the cause of contamination by leakage of the discharged solution to outside due to splashing or the like.

After that, supplying of compressed air is conducted. As a result of instruction from the control part 70, an air pump 43 is driven when an open/close valve 45 is a closed state and the open/close valve 45 opens. Now compressed air from an air pump 43 is supplied in a predetermined amount to the first (C1) cartridge 11 via a pressurizing nozzle 41.

Then, after the open/close valve 45 is closed, a pressurizing nozzle 41 is elevated by a nozzle ascending/descending motor 81 to drive the head transfer motor 26 whereupon the transfer head 40 is transferred to an extent of an arranged pitch of the cartridge 11. After that, a predetermined amount of compressed air is supplied similarly to the next second cartridge 11 (C2) (FIG. 6C).

In the sample solution S to which pressure is applied, nucleic acid is adsorbed and retained after passing through a nucleic acid-adsorptive porous membrane 11b while other liquid components are discharged to a waste solution container 12 from an outlet 11c of the lower end part. When all of the sample solution S passes through the nucleic acid-absorptive porous membrane 11b, pressure lowers below the pressure upon completion of the liquid discharge and finish of extraction of the cartridge 11 is detected by a pressure sensor 46. Said step is repeatedly conducted for the times of numbers of the cartridges 11.

Next is a washing treatment. The transfer head 40 is elevated after the above-mentioned supplying of the compressed air and returned onto the first cartridge (C1). Then a washing solution dispensing nozzle 51w of the transfer head 40 is stopped on the first cartridge (C1), a predetermined amount of the washing solution W is dispensed, the transfer head 40 is moved onto the next cartridge (C2) and the washing solution W is successively dispensed. When dispensing of the washing solution W onto all cartridges 11 finishes, the transfer head 40 is returned onto the first cartridge (C1).

Then, as shown in FIG. 6A to C, the pressurizing nozzle 41 of the transfer nozzle 40 descends and, after the lower end part of the pressurizing nozzle 41 is pressed and attached to the upper end opening of the cartridge 11 to close, the open/close valve 45 is opened as same as above and compressed air is supped to the cartridge 11. The washing solution W to which pressure is made to act conducts washing and removal of impurities other than nucleic acid passing through a nucleic acid-adsorptive porous membrane 11b and the washing solution W is discharged to a waste solution container 12 from the outlet 11c of the lower end. When all of the washing solution W in all of the cartridges 11 is discharged after passing through the nucleic acid-adsorptive porous membrane 11b, the transfer head 40 is made to act to the initial position. Incidentally, when the washing treatment is repeatedly conducted for two or more times, the above operation is repeated.

Next is a recovering treatment. Firstly, a rack 6 descends by an ascending/descending motor 47 synchronizing to the returning operation of the above-mentioned transfer head 40 after the washing treatment, the outlet 11c at lower end of the cartridge 11 is detached from the waste solution container 12 and, after that, an operation material 31 of the retaining mechanism 3 is transferred by driving of a container-exchanging motor 32 so that the rack is moved backward. As such, exchange of containers by which a recovering contains 13 is positioned below the cartridge 11 is conducted.

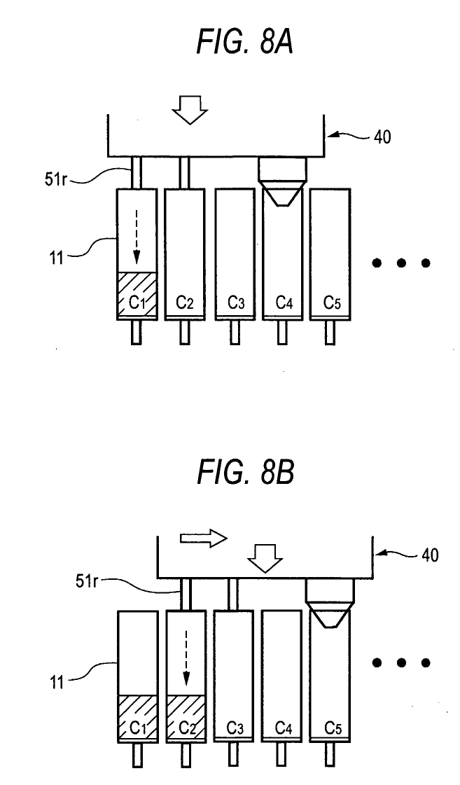

After that, the rack 6 is elevated by an ascending/descending motor 47 and the state where the lower end of the cartridge 11 is inserted into the recovering container 13 is retained. Then, as shown in FIG. 8A, the transfer head 40 is transferred whereby a nozzle 51r for dispensing the recovering solution is stopped on the first cartridge (C1) to dispense a predetermined amount of the recovering solution R, then the transfer head 40 is transferred to the next cartridge (C2) and dispensing of the recovering solution R is conducted successively (FIG. 8B). When dispensing of the recovering solution R to all cartridges 11 finishes, supplying of compressed air as same as above is conducted to each of the cartridges 11 as shown in FIG. 6A to C.

The recovering solution R where the compressed air is supplied the same as above and pressure is made to act passes through a nucleic acid-adsorptive porous membrane 11b, releases nucleic acid adsorbed there and nucleic acid is discharged to the recovering container 13 from the outlet 11c of the lower end together with the recovering solution R. When all recovering solution R in all of the cartridges 11 is discharged to the recovering container 13, the transfer head 40 transfers to the first dugout position immediately on the waste liquid container 57 whereupon a series of operations finishes.

The rack 6 where an extraction operation finishes is descended by driving the ascending/descending motor 47 and the cartridges 11 and the waste solution container 12 are taken out from the cartridge holder 21 and the rack 6 and discarded while the recovering container 13 is taken out from the rack 6, covered if necessary and subjected to the next nucleic acid analysis treatment.

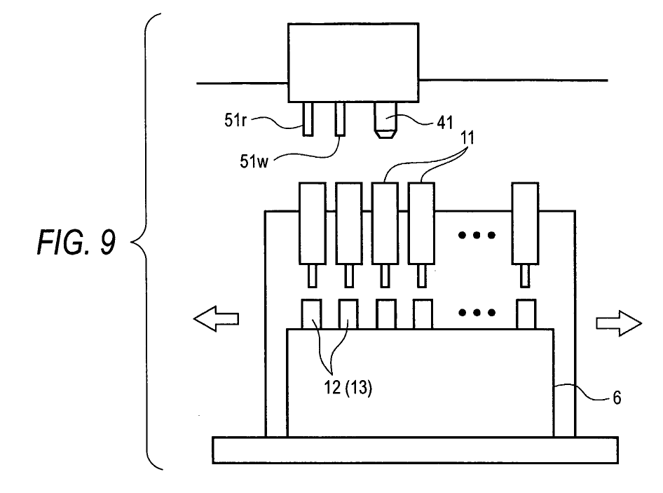

In the present embodiment, there was shown a constitution where plural cartridges 11 are supported on the fixed side while pressurizing nozzle 41 of a compressed air supplying mechanism 4 is supported in a freely-transferable manner along the arranged direction of the cartridges 11. However, the present invention is not limited to such a one but, as shown in FIG. 9 for the outline constitution of the nucleic acid-extracting apparatus, a constitution where a pressurizing nozzle 41 of the compressed air supplying mechanism 4 is supported at the fixed side and each of plural cartridges 11, waste solution container 12 and recovering container 13 are supported in a freely-transferable manner along the arranged direction thereof is acceptable as well. According to the constitution as shown in FIG. 9, treatment of a large amount of nucleic acid is able to be continuously conducted in such a manner that cartridge 11, waste solution container 12 and recovering container 13 each is continuously supplied to each of fixed pressurizing nozzle 41, washing solution-dispensing nozzle 51w and recovering solution-dispensing nozzle 51r and that where extraction of nucleic acid finishes is recovered.

Air which is supplied from an air pump 43 to a cartridge 11 may be any other gas so far as it does not affect the properties of sample solution, washing solution, liquid of recovered solution, etc.

As compared with the constitution where plural cartridges 11 are sucked together, it is possible in the nucleic acid-extracting apparatus 100 to control air supplying time, air supplying amount, etc. which are optimum to each cartridge 11. In addition, even when there are imbalances in liquid amount, viscosity, etc. of the sample solution, affection by them is hardly available, beat of extracting treatment of nucleic acid is able to be made quick, object for extraction of nucleic acid is able to be made broad and applicability of the apparatus is able to be enhanced. For example, in a constitution where supplying of air is conducted to plural cartridges at the same time, even when supplying of compressed air to a part of cartridges finishes, it is not possible to finish supplying of compressed air in case supplying of compressed air to other cartridges does not finish. Thus, until all of plural cartridges 11 finish, it is not possible to move to the next step. However, in accordance with the nucleic acid-extracting apparatus 100 of the present embodiment, each cartridge is subjected to a treatment successively and, therefore, there is no affection by other cartridges whereupon it is now possible that the optimum treatment is conducted within the shortest time.

In addition, since air is supplied to only one cartridge 11, ability of the air pump 43 is able to be made low as compared with the case where air is supplied to plural cartridges 11 at the same time. Accordingly, even an air pump 43 in a small size is able to be used, space for installing it is small and a compact constitution is achieved.

Further, as a result of the fact that cartridges 11 are arranged in a uniform arranging pitch and an arranging pitch of dispensing nozzles 51r, 51w to the pressurizing nozzle 41 is made integral multiple of the arranging pitch of a cartridge 11, it is now possible that dispensing treatment and compressing treatment are able to be conducted to plural different cartridges 11 at the same time whereby shortening of the treating time is achieved. Incidentally, cartridge 11, waste solution container 12 and recovering container 13 are also able to be arranged in curve such as in circular arc in addition to the linear arrangement. When they are arranged in circular arc for example, supplying and dispensing of compressed air are able to be done to each position on the circle using an arm where supporting axis is a center.

In the nucleic acid-extracting method and apparatus according to the present invention, at least one of time where a washing solution is dispensed and allowed to stand and time where a recovering solution is dispensed is controlled to a predetermined time.

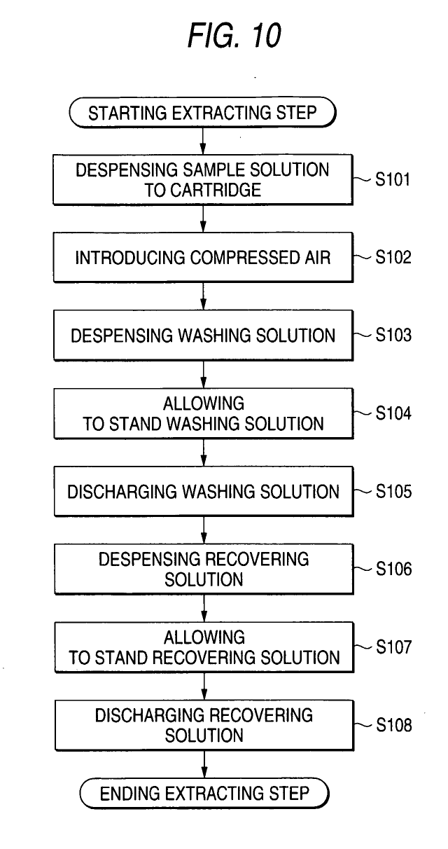

FIG. 10 is a flow chart which shows a process of the steps for extracting nucleic acid according to the present invention. In the nucleic acid-extracting steps which will be mentioned hereinafter, the already-mentioned nucleic acid-extracting apparatus 100 will be used.

As shown in FIG. 10, a sample solution S containing nucleic acid is firstly infused into a cartridge 11 (step S 101). Compressed air is introduced into the cartridge 11 where the sample solution S is received (step S 102) and a washing solution W is infused thereinto (step S 103). After dispensing the washing solution W, it is allowed to stand as it is before introduction of compressed air into a cartridge whereby a washing solution W into which nucleic acid is infused for a predetermined period is allowed to stand (step S 104). After said predetermined time elapses, the washing solution W is discharged into a waste solution container 12 (step S 105).

It is preferred that time for being allowed to stand the washing solution W is controlled to a range of 50 seconds to 1,000 seconds and it is more preferred to control to a range of 100 seconds to 300 seconds.

After dispensing the washing solution W, the washing solution W into which nucleic acid is dispensed for a predetermined time before introduction of compressed air into a cartridge is allowed to stand whereupon amount of nucleic acid extracted after the recovering treatment is able to be increased.

After that, the recovering solution R is recovered (step S 106). After dispensing the recovering solution R, a recovering solution R into which nucleic acid is dispensed for a predetermined time by being allowed to stand before introduction of compressed air to a cartridge 11 is allowed to stand (step S 107). After said predetermined time elapses, the recovering solution R is discharged to a recovering container 13 (step S 108).

It is preferred that time for being allowed to stand the recovering solution R is controlled to a range of 20 seconds to 300 seconds and it is more preferred to control to a range of 25 seconds to 60 seconds.

After dispensing the recovering solution R, the recovering solution R into which nucleic acid is dispensed for a predetermined time before introduction of compressed air into a cartridge is allowed to stand whereupon amount of nucleic acid extracted after the recovering treatment is able to be increased.

Time for dispensing and being allowed to stand the washing solution W or time for dispensing and being allowed to stand the recovering solution R is controlled to a predetermined time, there is achieved an effect that amount of the extractable nucleic acid is increased. When both of the time for dispensing and being allowed to stand the washing solution W and the time for dispensing and being allowed to stand the recovering solution R are controlled to a predetermined time each, it is now possible to further increase the amount of the extractable nucleic acid.

For example, when only the washing solution W is allowed to stand for a predetermined time, a procedure for being allowed to stand the recovering solution R as shown in the step S 107 in FIG. 10 is omitted. On the other hand, when only the recovering solution R is allowed to stand for a predetermined time, a procedure for being allowed to stand the washing solution W as shown in the step S 104 in FIG. 10 is omitted.

By referring to FIG. 3, a mechanism where each of the time for dispensing and being allowed to stand the washing solution and the time for dispensing and being allowed to stand the recovering solution are controlled to a predetermined time will be illustrated.

As shown in FIG. 3, the control part 70 controls air pump 43, relief valve 44 and pressurizing nozzle 41 so that, after dispensing of the washing solution W in the washing treatment, compressed air is not provided to a cartridge 11. Then, nucleic acid is dipped in the washing solution W for a predetermined time in the cartridge 11, compressed air is sent again by controlling air pump 43, relief valve 44 and pressurizing nozzle 41 by a controlling part 70 and the washing solution W is discharged from the cartridge 11. Incidentally, the control part 70 controls air pump 43, relief valve 44 and pressurizing nozzle 41 after dispensing the recovering solution R during the recovering treatment so that compressed air is not sent to the cartridge 11. Nucleic acid is dipped in the recovering solution R for a predetermined time in the cartridge and, again, compressed air is sent by controlling air pump 43, relief valve 44 and pressurizing nozzle 41 by a control part 70 whereupon the recovering solution R is discharged from the cartridge. In this embodiment, the control part 70 functions as a means for controlling the time for being allowed to stand.

In this embodiment, there is used a mechanism where each of the time for dispensing and being allowed to stand the washing solution W and the time for dispensing and being allowed to stand the recovering solution R are controlled to predetermined time is conducted by a control part 70 although that is non-limitative. For example, there may be installed an air supplying stopper which cooperates with air pump 43, relief valve 44 and pressurizing nozzle 41 for stopping the supply of compressed air so that, after dispensing the washing solution W and the recovering solution R are dispensed, each of them is allowed to stand in the cartridge for a predetermined time.

Now, the nucleic acid-adsorptive porous membrane (nucleic acid-adsorptive porous material) 11b which is installed in the above cartridge 11 will be illustrated in detail.

The nucleic acid-adsorptive porous membrane 11b contained in the above cartridge 11 is fundamentally a porous substance through which nucleic acid is able to pass. Its surface has a characteristic that nucleic acid in the sample solution is adsorbed by means of chemical bonding force and is constituted in such a manner that, upon washing by a washing solution, the adsorbed state is retained and, upon recovering by a recovering solution, adsorptive force of nucleic acid is made weak for releasing.

The nucleic acid-adsorbing porous membrane 11b contained in the above cartridge 11 is preferably a porous membrane to which nucleic acids adsorb based on an interaction wherein ion bond does not substantially participate. This means that no “ionization” takes place under the condition of using the porous membrane, and it is surmised that nucleic acids and the porous membrane pull against each other due to change in surrounding polarity. Thus, nucleic acids can be separated and purified with excellent separating performance and good washing efficiency. Preferably, the nucleic acid-adsorbing porous membrane is a porous membrane having a hydrophilic group, and it is surmised that hydrophilic group of nucleic acids and hydrophilic group of the porous membrane come to pull against each other when the surrounding polarity is changed.

Here, the term “porous membrane having a hydrophilic group” means a porous membrane wherein the material constituting the porous membrane itself has the hydrophilic group, or a porous membrane obtained by treating or coating a porous membrane-constituting material in order to introduce the hydrophilic group into the porous membrane. The porous membrane-constituting material may be an organic or inorganic material. For example, there may be used a porous membrane wherein the porous membrane-constituting material itself is an organic material having a hydrophilic group, a porous membrane which is obtained by treating a porous membrane made of a hydrophilic group-free organic material so as to introduce the hydrophilic group thereinto, a porous membrane obtained by coating a porous membrane made of a hydrophilic group-free organic material with a material having a hydrophilic group to thereby introduce the hydrophilic group, a porous membrane wherein the porous membrane-constituting material itself is an inorganic material having a hydrophilic group, a porous membrane which is obtained by treating a porous membrane made of a hydrophilic group-free inorganic material so as to introduce the hydrophilic group thereinto, and a porous membrane obtained by coating a porous membrane made of a hydrophilic group-free inorganic material with a material having a hydrophilic group to thereby introduce the hydrophilic group. In view of processing ease, it is preferable to use an organic material such as an organic polymer as the material for constituting the porous membrane.

The hydrophilic group means a polar group (atoms) capable of exerting an interaction with water, and includes all groups (atoms) participating in adsorption of nucleic acid. As the hydrophilic group, those which exhibit about a middle level of interaction with water (see, “group having not so strong hydrophilicity” in the item of “hydrophilic group” described in Kagaku Dai-jiten, published by Kyoritsu Shuppan) are preferred, and examples thereof include a hydroxyl group, a carboxyl group, a cyano group and a hydroxyethyl group, with a hydroxyl group being preferred.

With regard to the porous membrane having a hydrophilic group which is able to be used in the present invention, porous membrane of an organic material having amide group may be exemplified. A polyamide is able to be preferably used as the organic material having amide group. Examples of the polyamide are fibroin, polyamino acid, polypeptide, polyacrylamide, Nylon 46, Nylon 66, Nylon 610, Nylon 612, Nylon 6, Nylon 7, Nylon 11 and Nylon 12 although they are non-limitative. It is also possible to use modified Nylon such as N-methyl modified Nylon, N-alkoxymethyl modified Nylon and N-alkylthiomethyl modified Nylon. With regard to a porous membrane of a polyamide, those which are produced from the materials and by the process mentioned, for example, in U.S. Pat. Nos. 2,783,894, 3,408,315, 4,340,479, 4,340,480 and 4,450,126, German Patent No. 3,138,525 and Japanese Patent Laid-Open No. 58/037,842 may be used although they are non-limitative.

With regard to the porous membrane of an organic material having hydroxyl group which is able to be used in the present invention, its examples includes porous membrane formed by polyhydroxyethylacrylic acid, polyhydroxyethylmethacrylic acid, polyvinyl alcohol, polyvinylpyrrolidone, polyacrylic acid, polymethacrylic acid and polysaccharide such as polyoxyethylene and acetylcellulose acetylcellulose mixture having different acetyl values and porous membrane of an organic material having a polysaccharide structure is able to be used particularly preferably.

With regard to organic material for porous membrane, it is also possible to use a saponified product of polymer vinyl acylate and a saponified copolymer of two or more kinds of monomers containing a monomer unit of at least vinyl acylate preferably. It is preferred that saponification degree of the saponified product of polymer vinyl acylate and the saponified copolymer of two or more kinds of monomers containing a monomer unit of at least vinyl acylate is 1% or more. It is also preferred that an acyl group of the above vinyl acylate is selected from at least one of acetyl group, propionyl group, butyroyl group, valery group, heptanoyl group, octanoyl group, decanoyl group, dodecanoyl group, tridecanoyl group, hexadecanoyl group and octadecanoyl group.

As the organic material having a polysaccharide structure, cellulose, hemicellulose, dextran, amylase, amylopectin, starch, glycogen, pullulan, mannan, glucomannan, lichenan, isolichenan, laminaran, carrageenan, xylan, fructan, alginic acid, hyaluronic acid, chondroitin, chitin and chitosan can preferably be used. However, these are not limitative, and any organic material having a polysaccharide structure of its derivative may be used. Also, an ester derivative of any of these polysaccharides can preferably be used. Further, a saponification product of the ester derivative of any of these polysaccharides can preferably be used.

As the ester of the ester derivative of any of the above-mentioned polysaccharides, one or more members selected from among carboxylates, nitrates, sulfates, sulfonates, phosphates, phosphonates and pyrophosphates are preferably selected. Also, saponification products of the carboxylates, nitrates, sulfates, sulfonates, phosphates, phosphonates and pyrophosphates can more preferably be used.

As the carboxylates of any of the above-mentioned polysaccharides, one or more members selected from among alkylcarbonyl esters, alkenylcarbonyl esters, aromatic carbonyl esters and aralkylcarbonyl esters are preferably selected. Also, saponification products of the alkylcarbonyl esters, alkenylcarbonyl esters, aromatic carbonyl esters and aralkylcarbonyl esters of any of the above-mentioned polysaccharides can more preferably be used.

As the ester group of the alkylcarbonyl esters of any of the above-mentioned polysaccharides, one or more members selected from among an acetyl group, a propionyl group, a butyroyl group, a valeryl group, a heptanoyl group, an octanoyl group, a decanoyl group, a dodecanoyl group, a tridecanoyl group, a hexadecanoyl group and an octadecanoyl group are preferably selected. Also, saponification products of any of the above-mentioned polysaccharides having one or more ester groups selected from among an acetyl group, a propionyl group, a butyloyl group, a valeryl group, a heptanoyl group, an octanoyl group, a decanoyl group, a dodecanoyl group, a tridecanoyl group, a hexadecanoyl group and an octadecanoyl group can more preferably be used.

As the ester group of the alkenylcarbonyl esters of any of the above-mentioned polysaccharides, one or more of an acryl group and a methacryl group are preferably selected. Also, saponification products of any of the above-mentioned polysaccharides having ester groups of one or more of an acyl group and a methacryl group can more preferably be used.

As the ester group of the aromatic carbonyl esters of any of the above-mentioned polysaccharides, one or more of a benzoyl group and a naphthaloyl group are preferably selected. Also, saponification products of any of the above-mentioned polysaccharides having ester groups of one or more of a benzoyl group and a naphthaloyl group can more preferably be used.

As the nitrates of any of the polysaccharides, nitrocellulose, nitrohemicellulose, nitrodextran, nitroagarose, nitrodextrin, nitroamylase, nitroamylopectin, nitroglycogen, nitropullulan, nitromannan, nitroglucomannan, nitrolichenan, nitroisolichenan, nitrolaminaran, nitrocarrageenan, nitroxylan, nitrofructan, nitroalginic acid, nitrohyaluronic acid, nitrochondroitin, nitrochitin and nitrochitosan can preferably be used.

Also, saponification products of nitrocellulose, nitrohemicellulose, nitrodextran, nitroagarose, nitrodextrin, nitroamylase, nitroamylopectin, nitroglycogen, nitropullulan, nitromannan, nitroglucomannan, nitrolichenan, nitroisolichenan, nitrolaminaran, nitrocarrageenan, nitroxylan, nitrofructan, nitroalginic acid, nitrohyaluronic acid, nitrochondroitin, nitrochitin and nitrochitosan can more preferably be used.

As the sulfates of any of the polysaccharides, cellulose sulfate, hemicellulose sulfate, dextran sulfate, agarose sulfate, dextrin sulfate, amylase sulfate, amylopectin sulfate, glycogen sulfate, pullulan sulfate, mannan sulfate, glucomannan sulfate, lichenan sulfate, isolichenan sulfate, laminaran sulfate, carrageenan sulfate, xylan sulfate, fructan sulfate, alginic acid sulfate, hyaluronic acid sulfate, chondroitin sulfate, chitin sulfate and chitosan sulfate can preferably be used.

Also, saponification products of cellulose sulfate, hemicellulose sulfate, dextran sulfate, agarose sulfate, dextrin sulfate, amylase sulfate, amylopectin sulfate, glycogen sulfate, pullulan sulfate, mannan sulfate, glucomannan sulfate, lichenan sulfate, isolichenan sulfate, laminaran sulfate, carrageenan sulfate, xylan sulfate, fructan sulfate, alginic acid sulfate, hyaluronic acid sulfate, chondroitin sulfate, chitin sulfate and chitosan sulfate can more preferably be used.

As the sulfonates of any of the aforementioned polysaccharides, one or more members selected from among alkyl sulfonates, alkenyl sulfonates, aromatic sulfonates and aralkyl sulfonates are preferably selected. Also, saponification products of alkyl sulfonates, alkenyl sulfonates, aromatic sulfonates and aralkyl sulfonates of any of the above-mentioned polysaccharides can more preferably be used.

As the phosphates of any of the aforementioned polysaccharides, cellulose phosphate, hemicellulose phosphate, dextran phosphate, agarose phosphate, dextrin phosphate, amylase phosphate, amylopectin phosphate, glycogen phosphate, pullulan phosphate, mannan phosphate, glucomannan phosphate, lichenan phosphate, isolichenan phosphate, laminaran phosphate, carrageenan phosphate, xylan phosphate, fructan phosphate, alginic acid phosphate, hyaluronic acid phosphate, chondroitin phosphate, chitin phosphate and chitosan phosphate can preferably be used.

Also, saponification products of cellulose phosphate, hemicellulose phosphate, dextran phosphate, agarose phosphate, dextrin phosphate, amylase phosphate, amylopectin phosphate, glycogen phosphate, pullulan phosphate, mannan phosphate, glucomannan phosphate, lichenan phosphate, isolichenan phosphate, laminaran phosphate, carrageenan phosphate, xylan phosphate, fructan phosphate, alginic acid phosphate, hyaluronic acid phosphate, chondroitin phosphate, chitin phosphate and chitosan phosphate can more preferably be used.

As the phosphonates of any of the aforementioned polysaccharides, cellulose phosphonate, hemicellulose phosonphate, dextran phosphonate, agarose phosphonate, dextrin phosphonate, amylase phosphonate, amylopectin phosonphate, glycogen phosphonate, pullulan phosphonate, mannan phosphonate, glucomannan phosphonate, lichenan phosphonate, isolichenan phosphonate, laminaran phosphonate, carrageenan phosphonate, xylan phosphonate, fructan phosphonate, alginic acid phosphonate, hyaluronic acid phosphonate, chondroitin phosphonate, chitin phosphonate and chitosan phosphonate can preferably be used.

Also, saponification products of cellulose phosphonate, hemicellulose phosphonate, dextran phosphonate, agarose phosphonate, dextrin phosphonate, amylase phosphonate, amylopectin phosphonate, glycogen phosphonate, pullulan phosphonate, mannan phosphonate, glucomannan phosphonate, lichenan phosphonate, isolichenan phosphonate, laminaran phosphonate, carrageenan phosphonate, xylan phosphonate, fructan phosphonate, alginic acid phosphonate, hyaluronic acid phosphonate, chondroitin phosphonate, chitin phosphonate and chitosan phosphonate can more preferably be used.

As the pyrophosphates of any of the aforementioned polysaccharides, cellulose pyrophosphate, hemicellulose pyrophosphate, dextran pyrophosphate, agarose pyrophosphate, dextrin pyrophosphate, amylase pyrophosphate, amylopectin pyrophosphate, glycogen pyrophosphate, pullulan pyrophosphate, mannan pyrophosphate, glucomannan pyrophosphate, lichenan pyrophosphate, isolichenan pyrophosphate, laminaran pyrophosphate, carrageenan pyrophosphate, xylan pyrophosphate, fructan pyrophosphate, alginic acid pyrophosphate, hyaluronic acid pyrophosphate, chondroitin pyrophosphate, chitin pyrophosphate and chitosan pyrophosphate can preferably be used.

Also, saponification products of cellulose pyrpphosphate, hemicellulose pyrophosphate, dextran pyrophosphate, agarose pyrophosphate, dextrin pyrophosphate, amylase pyrophosphate, amylopectin pyrophosphate, glycogen pyrophosphate, pullulan pyrophosphate, mannan pyrophosphate, glucomannan pyrophosphate, lichenan pyrophosphate, isolichenan pyrophosphate, laminaran pyrophosphate, carrageenan pyrophosphate, xylan pyrophosphate, fructan pyrophosphate, alginic acid pyrophosphate, hyaluronic acid pyrophosphate, chondroitin pyrophosphate, chitin pyrophosphate and chitosan pyrophosphate can more preferably be used.

As the ether derivatives of any of the aforementioned polysaccharides, methyl cellulose, ethyl cellulose, carboxymethyl cellulose, carboxyethyl cellulose, carboxyethyl-carbamoylethyl cellulose, hydroxymethyl cellulose, hydroxyethyl cellulose, hydroxypropyl cellulose, hydroxypropylmethyl cellulose, hydroxyethylmethyl cellulose, cyanoethyl cellulose and carbamoylethyl cellulose can be used, though the ether derivatives not being limited thereto. It is preferable to use hydroxymethyl cellulose or hydroxyethyl cellulose.

Those wherein hydroxyl groups of any of the polysaccharides are halogenated with any substitution degree can also be preferably used.

The above-mentioned cellulose ester derivative will be mentioned as hereunder. Examples of the cellulose which is a material for the above-mentioned cellulose ester derivative are natural cellulose such as cotton linter and wood pulp (pulp from broad leaf trees and pulp from needles trees), hemp and cellulose produced during the incubating process of acetic acid bacteria and that where the above is subjected to acid hydrolysis, mechanical disintegration, explosive treatment or extruding treatment at high temperature so that degree of polymerization is adjusted. Any cellulose ester derivative produced from any material cellulose may be used and, in some cases, it may be used after mixing. Detailed description for such cellulose materials is, for example, found in “Plastic Materials (17), Cellulose-Type Fibers” (by Marusawa and Uda; published by Nikkan Kogyo Shimbunsha in 1970).

According to the above, molecular weight of cellulose has a broad range. For example, natural cellulose is 600,000 to 1,500,000 (degree of polymerization: ca. 3,500 to 10,000), pure linter is 80,000 to 500,000 (degree of polymerization: ca. 500 to 3,000) and wood pulp is 80,000 to 1,340,000 (degree of polymerization: ca. 500 to 2,100). Here, molecular weight greatly affects the mechanical strength of cellulose or derivatives thereof and, when molecular weight becomes small, the mechanical strength suddenly lowers as from a specific degree of polymerization although it is able to be used without any problem as a material for the nucleic acid-adsorptive porous membrane of the present invention.

As an example of the above-mentioned nucleic acid-adsorptive porous membrane, porous membrane of a cellulose ester derivative produced by esterification of cellulose is able to be used. However, the particularly preferred above-mentioned cellulose is not able to be used as it is but is used as pure linter or pure high-class wood pulp which is produced by purification of linter or pulp. Linter is a short fiber having short fiber length in cotton fibers of cottonseed, contains high amount of α-cellulose (such as 88 to 92% by weight) and has high purity containing little impurities. Pure linter is able to be prepared when crude linter is subjected to removal of dust, steam boiling with alkali, bleaching, treatment with acid, dehydration and drying. Details are mentioned in pages 25 to 28 of “Plastic Materials (17), Cellulose-Type Resins (by Marusawa and Uda; published by Nikkan Kogyo Shimbunsha in 1970) and, in Tables 2 to 3 thereof, characteristics are mentioned. More preferred pure linter is produced by the present invention.

Pure pulp is also mentioned in pages 28 to 32 of the same book, characteristics are also mentioned in Tables 2 to 4 thereof and pulp which is purified by said means is preferred as a material for cellulose ester derivative as well. Here, it is also preferred to use by mixing the purified cotton wool linter with wood pulp and, although their mixing ratio is not particularly limited, it is preferred to be 5/95 to 95/5 and, more preferably, 10/90 to 90/10. As a result of mixing, solubility is enhanced whereby surface property and dynamic characteristic of the porous membrane of cellulose ester derivative are able to be improved.

Among the above, α-cellulose content which is an index for purity of the pulp is able to be selected, for example, from the range of 80 to 100% by weight and, in the case of wood pulp, it is usually about 85 to 98%. In the present invention, it is also possible to use a low-purity pulp such as pulp where content of α-cellulose is 80 to 96% (particularly, 92 to 96%). Among the pulp as such, wood pulp is usually used.

Further, in the nucleic acid-adsorptive porous membrane of the present invention, although glucose is a main component in neutral constituting saccharide components in pulp or cotton wool, mannose and xylose may be contained as well. Although there is no particular limitation for its ratio, the molar ratio of mannose/xylose is 0.35/1 to 3.0/1, preferably 0.35/1 to 2.5/1 and, more preferably, 0.35/1 to 2 μl. In the cellulose triacetate produced at that time, total amount of mannose and xylose is 0.01 to 5 molar % and, preferably, 0.1 to 4 molar %. Incidentally, “mannose” and “xylose” are main constituting saccharides for hemicellulose (xylan, glucomannan, etc.) contained in pulp. The constituting saccharide components for those material pulps and the resulting cellulose ester derivative (cellulose triacetate) are able to be specifically analyzed by a method mentioned in Japanese Patent Laid-Open No. 11/130,301.

With regard to a porous membrane of cellulose, a porous membrane of regenerated cellulose may be preferably used. Examples of the regenerated cellulose are a product where surface of or all of solid of acetylcellulose is made into cellulose by a saponifying treatment, a product from a copper ammonia solution of cellulose, a product from a viscose solution of cellulose and a product from an alkaline solution of cellulose and they are different from natural cellulose in view of crystalline state, etc. In cellulose, there are crystal types of I, II, III and IV and, in the present invention, any crystal type may be preferably used or each of the crystal types I, II, III and IV may be contained in any proportion. With regard to porous membrane of regenerated cellulose produced from porous membrane of acetylcellulose, it is possible to use that which is produced using materials and methods mentioned in Japanese Patent Publication No. 45/4,633, Japanese Patent Laid-Open No. 56/100,604, etc. although they are non-limitative. With regard to porous membrane of regenerated cellulose produced from a copper ammonia solution of cellulose, it is possible to use that which is produced using materials and methods mentioned, for example, in Japanese Patent Laid-Open Nos. 58/089,625, 58/089,626, 58/089,627, 58/089,628, 59/045,333, 59/045,334, 59/199,728, 61/274,707, 62/001,403, 63/161,927 and 07/330,945 although they are non-limitative. It is also possible to produce a regenerated cellulose porous membrane in the similar manner from a viscose solution produced by the reaction of cellulose with alkali and carbon disulfide where material composition and aggregating method are modified and the product is able to be used in the present invention. With regard to the regenerated cellulose porous membrane produced from an alkaline solution of cellulose, that which is produced using materials and methods mentioned, for example, in Japanese Patent Laid-Open Nos. 62/240,328, 62/240,329 and 01/188,539 may be also used although they are non-limitative.

In the nucleic acid-adsorptive porous membrane of the present invention, it is preferred that a viscosity-average degree of polymerization of the above cellulose ester derivative is 200 to 3,000. It is preferred that the ratio of a weight-average molecular weight of the above cellulose ester derivative to a number-average molecular weight of the above cellulose ester derivative is 0.8 to 2. It is preferred that the above cellulose ester derivative contains an acid where acid dissociation index is 1.93 to 4.5 or a salt thereof.

It is preferred that, in the above cellulose ester derivatives, residual acetic acid amount or fatty acid of C₃₋₂₂ is 0.5% by weight or less. It is preferred that the cellulose ester derivative contains 1 ppb to 10,000 ppm of at least one member of alkali metal and/or alkaline earth metal. It is preferred that the cellulose acylate contains 1 ppb to 1,000 ppm of at least one member of aluminum, bismuth, silicon and heavy metals (such as chromium, manganese, iron, cobalt, nickel, copper, zinc, arsenic, silver, cadmium, tin, antimony, gold, platinum, mercury and lead).

With regard to the particularly preferred porous membrane of cellulose ester derivative, a porous membrane of an organic macromolecular substance comprising acetylcelluloses having different acetyl values may be listed. With regard to a mixture of acetylcelluloses having different acetyl values, a mixture of triacetylcellulose and diacetylcellulose, a mixture of triacetylcellulose and minoacetylcellulose, a mixture of triacetylcellulose and diacetylcellulose and a mixture of diacetylcellulose and monoacetylcellulose may be preferably used. A mixture of triacetylcellulose and diacetylcellulose is used particularly preferably. It is preferred that a mixing ratio (ratio by weight) of a mixture of triacetylcellulose and diacetylcellulose is 99:1 to 1:99 and, more preferably, 90:10 to 50:50.

With regard to the above-mentioned cellulose ester derivative, it is also preferred to utilize the cellulose ester derivatives mentioned in Japanese Patent Laid-Open Nos. 10/045,803, 11/269,304, 08/231,761,08/231,761, 10/060,170, 09/040,792, 11/005,851, 11/269,304,09/090,101, 57/182,737,04/277,530, 11/292,989, 12/131,524, 12/137,115, etc. There is also no particular limitation for those materials in connection with the nucleic acid-adsorptive porous membrane of the present invention.

As a means for evaluating the structure of cellulose, X-ray analysis is used as well. According to that, there is described that cellulose molecule is arranged in parallel in the direction of fiber axis, pulled each other by hydrogen bond and forms a unit cell by a celluose unit of five cellulose molecules. According to an X-ray method, its degree of crystallization in the case of natural cellulose is about 70% and cellulose as such is also able to be used for the production of the cellulose ester derivative of the present invention.

With regard to the cellulose which is also utilized in the present invention, various analyses thereof have been conducted already and are mentioned in detail in ASTM Standard Part 15, TAPPI Standard (Technical Association of the Pulp and Paper Industry), JIS P 8101, etc. Examples of the items to be measured are ash, amounts of calcium oxide and magnesium oxide, α-cellulose, β-cellulose and copper value.

Herein, the saponification treatment means that acetyl cellulose comes in contact with saponification treatment solution (e.g., Sodium hydroxide solution). As a result, the saponification treatment solution contacted ester group of ester derivative of acetyl cellulose is hydrolyzed, and a hydroxyl group is introduced to form regenerated cellulose. Thereby the prepared regenerated cellulose is different in crystalline form from the original cellulose. In order to change the surface saponification degree, saponification treatment is conducted having changed the concentration or treating time of sodium hydroxide. The surface saponification degree is determined by means of NMR, IR or XPS (e.g., detecting a degree of reduction in the peak of carbonyl group).

An example of porous membrane of an organic material having a polysaccharide structure is a surface saponified product of acetylcellulose mentioned in Japanese Patent Laid-Open No. 2003/128,691. The surface-saponified product of acetylcellulose is a product where a mixture of acetylcelluloses having different acetyl values is subjected to a saponifying treatment and preferably used ones thereof are a saponified product of a mixture of triacetylcellulose and diacetylcellulose, a saponified product of a mixture of triacetylcellulose, diacetylcellulose and monoacetylcellulose and a saponified product of a mixture of diacetylcellulose and monoacetylcellulose. It is preferred that a mixing ratio (ratio by weight) of a mixture of triacetylcellulose and diacetylcellulose is 99:1 to 1:99. It is more preferred that the mixing ratio of a mixing ratio of triacetylcellulose and diacetylcellulose is 90:10 to 50:50. In that case, amount (density) of hydroxyl groups on the surfaced of solid phase may be able to be controlled by the degree of oxidizing treatment (saponifying rate). In order to enhance the separating efficiency of nucleic acid, the more the amount (density) of hydroxyl groups, the better. For example, in the case of acetylcellulose such as triacetylcellulose, saponifying rate (surface saponifying rate) is preferably about 5% or more and, more preferably, it is 10% or more. In order to make the surface area of the organic macromolecular substance having hydroxyl groups large, it is preferred that porous membrane of acetylcellulose is subjected to a saponifying treatment. In that case, when a porous membrane where surface and back are symmetric is used, there is an advantage that production is possible without discrimination of surface and back of the membrane while, when a porous membrane where surface and back are asymmetric is used, there is an advantage that risk of clogging of pores can be reduced whereby that is preferably used.

A method for introducing a hydroxyl group to a porous membrane comprising organic material not having a hydroxyl group is to bond a graft polymer chain having a hydroxyl group in inner polymer strand or a side chain to a porous membrane.

A method for bonding a graft polymer chain to an organic material of a porous membrane include two methods such as a method for chemically bonding a porous membrane with graft polymer chain, and a method for polymerizing a compound having a double bond capable of polymerization using a porous membrane as a starter to form graft polymer chain.

Firstly, in the method in which the porous membrane and graft polymer chain are chemically bonded, a polymer having a functional group capable of reacting with the porous membrane in the terminus or side chain of the polymer is used, and they are grafted through a chemical reaction of this functional group with a functional group of the porous membrane. The functional group capable of reacting with the porous membrane is not particularly limited with the proviso that it can react with a functional group of the porous membrane, and its examples include a silane coupling group such as alkoxysilane, isocyanate group, amino group, hydroxyl group, carboxyl group, sulfonate group, phosphate group, epoxy group, allyl group, methacryloyl group, acryloyl group and the like.

Examples of the compound particularly useful as the polymer having a reactive functional group in the terminus or side chain of the polymer include a polymer having trialkoxysilyl group in the polymer terminus, a polymer having amino group in the polymer terminus, a polymer having carboxyl group in the polymer terminus, a polymer having epoxy group in the polymer terminus and a polymer having isocyanate group in the polymer terminus. The polymer to be used in this case is not particularly limited with the proviso that it has a hydrophilic group which is concerned in the adsorption of nucleic acid, and its illustrative examples include polyhydroxyethyl acrylic acid, polyhydroxyethyl methacrylic acid and salts thereof, polyvinyl alcohol, polyvinyl pyrrolidone, polyacrylic acid; polymethacrylic acid and salts thereof, polyoxyethylene and the like.

The method in which a compound having a polymerizable double bond is made into a graft polymer chain by polymerizing it using the porous membrane as the starting point is generally called surface graft polymerization. The surface graft polymerization method means a method in which an active species is provided on the base material surface by plasma irradiation, light irradiation, heating or the like method, and a polymerizable compound having double bond arranged in contact with a porous membrane is linked to the porous membrane by polymerization.

It is necessary that the compound useful for forming a graft polymer chain linked to the base material has both of two characteristics of having a polymerizable double bond and having a hydrophilic group which is concerned in the adsorption of nucleic acid. As such a compound, any one of the polymers, oligomers and monomers having a hydrophilic group can be used with the proviso that it has a double bond in the molecule. Particularly useful compound is a monomer having a hydrophilic group.

As illustrative examples of the particularly useful monomer having a hydrophilic group, the following monomers can be cited. For example, 2-hydroxyethyl acrylate, 2-hydroxyethyl methacrylate, glycerol monomethacrylate and the like hydroxyl group-containing monomers can be used particularly suitably. In addition, acrylic acid, methacrylic acid and the like carboxyl group-containing monomers or alkali metal salts and amine salts thereof can also be used suitably.

As another method for introducing a hydrophilic group into a porous membrane of an organic material having no hydrophilic group, a material having a hydrophilic group can be coated. The material to be used in the coating is not particularly limited with the proviso that it has a hydrophilic group which is concerned in the adsorption of nucleic acid, but is preferably a polymer of an organic material from the viewpoint of easy handling. Examples of the polymer include polyhydroxyethyl acrylate, polyhydroxyethyl methacrylate and salts thereof, polyvinyl alcohol, polyvinyl pyrrolidone, polyacrylic acid, polymethacrylic acid and salts thereof, polyoxyethylene, acetyl cellulose, a mixture of acetyl celluloses having different acetyl values and the like, but a polymer having a polysaccharide structure is desirable.

Alternatively, it is possible to coat acetyl cellulose or a mixture of acetyl celluloses having different acetyl values on a porous membrane of an organic material having no hydrophilic group and then to subject the coated acetyl cellulose or a mixture of acetyl celluloses having different acetyl values to a saponification treatment. In that case, the saponification ratio is preferably about 5% or more. The saponification ratio is more preferably 10% or more.

As the porous membrane of an inorganic material having a hydrophilic group, a porous membrane containing a silica compound can be exemplified. As the porous membrane containing a silica compound, a glass filter can be exemplified. Also can be exemplified is a porous silica thin membrane described in Japanese Patent No. 3,058,3442. This porous silica thin membrane can be prepared by spreading a developing solution of a cationic amphipathic substance having an ability to form a bimolecular membrane on a base material, preparing multi-layered bimolecular thin membranes of the amphipathic substance by removing the solvent from the liquid membrane on the base material, allowing the multi-layered bimolecular thin membranes to contact with a solution containing a silica compound, and then extracting and removing the aforementioned multi-layered bimolecular thin membranes.

Regarding the method for introducing a hydrophilic group into a porous membrane of an inorganic material having no hydrophilic group, there are a method in which the porous membrane and a graft polymer chain are chemically bonded and a method in which a graft polymer chain is polymerized using a hydrophilic group-containing monomer having a double bond in the molecule, using the porous membrane as the starting point.

When the porous membrane and graft polymer chain are attached by chemical bonding, a functional group capable of reacting with a terminal functional group of the graft polymer chain is introduced into an inorganic material, and the graft polymer chain is chemically bonded thereto. Also, when a graft polymer chain is polymerized using a hydrophilic group-containing monomer having a double bond in the molecule and using the porous membrane as the starting point, a functional group which becomes the starting point in polymerizing the double bond-containing compound is inserted into the inorganic material.

As the graft polymer having a hydrophilic group and hydrophilic group-containing monomer having a double bond in the molecule, the aforementioned graft polymer having a hydrophilic group and hydrophilic group-containing monomer having a double bond in the molecule, described in the foregoing regarding the method for introducing a hydrophilic group into a porous membrane of an organic material having no hydrophilic group, can be suitably use.

Another method for introducing a hydrophilic group to a porous membrane comprising inorganic material not having a hydrophilic group is to coat a material having a hydrophilic group thereon. Materials used in coating are not limited as long as the hydroxyl group participates in the adsorption of nucleic acid, but for easy workability, a polymer of organic material is preferred. Examples of polymer include polyhydroxyethylacrylate, polyhydroxyethylmethacrylate and their salts, polyvinyl alcohol, polyvinylpyrrolidone, polyacrylate, polymethacrylate and their salts, polyoxyethylene, acetyl cellulose, and a mixture of acetyl celluloses which are different in acetyl value from each other.

Examples of the porous membrane comprising inorganic material not having a hydrophilic group including aluminum and the like metals, glass, cement, pottery and the like ceramics, or a porous membrane fabricated by stepping new ceramics, silicon, active charcoal, etc.

To the porous membrane comprising inorganic material not having a hydrophilic group, acetyl cellulose or a mixture of acetyl celluloses which are different in acetyl value from each other is coated thereon, and the coated acetyl cellulose and a mixture of acetyl celluloses which are different in acetyl value from each other can be saponified. In this case, the surface saponification degree in a range of 5% or more is preferred. It is more preferred to have the surface saponification degree in a range of 10% or more.

In the above steps for the preparation of the nucleic acid-adsorptive solid carrier, various additives depending upon use (such as plasticizer, antistatic agent, deterioration preventer, ultraviolet preventer, surfactant, releasing agent, coloring agent, reinforcing agent and cross-linking agent) may be added. With regard to the stage for the addition, although it may be added in any of a dope-preparing step, it is also possible to add a step for adding the additive to the final preparing step in the dope-preparing step.

If necessary, the above-mentioned nucleic acid-adsorptive solid carrier may contain a plasticizer and the plasticizers such as phosphates and carboxylates mentioned in Japanese Patent Laid-Open No. 2002/265,636, polyhydric alcohols mentioned in Japanese Patent Laid-Open 02/006,826, (di)pentaethyrithol esters mentioned in Japanese Patent Laid-Open Nos. 05/194,788, 60/250,053, 04/227,941, 06/016,869, 05/271,471, 07/286,068, 05/005,047, 11/080,381, 07/020,317, 08/057,879, 10/152,568, 10/120,824 and 11/124,445, glycerol esters mentioned in Japanese Patent Laid-Open No. 11/246,704, diglycerol esters mentioned in Japanese Patent Laid-Open No. 2000/063,560, citrates mentioned in Japanese Patent Laid-Open No. 11/092,574, substituted phenylphosphates mentioned in Japanese Patent Laid-Open No. 11/090,946 and the plasticizers mentioned in Japanese Patent Laid-Open No. 56/100,604 may be preferably used. In accordance with those descriptions, there are many preferred descriptions not only for plasticizers but also for utilization method or characteristic thereof and they may be preferably used in the nucleic acid-adsorptive solid carrier of the present invention as well.

In order to prevent that the membrane is charged upon handling, antistatic agent may be added to the above-mentioned nucleic acid-adsorptive solid carrier. With regard to the antistatic agent, ionic conductive substance and conductive fine particles are preferably used. Here, an ionic conductive substance means a substance which shows electric conductivity and contains ion which is a carrier for carrying electricity and an example thereof is an ionic macromolecular compound. Examples of the ionic conductive substance are an anionic macromolecular substance mentioned in Japanese Patent Publication Nos. 49/23,826, 49/23,827 and 47/28,937; an ionene-type polymer having a dissociating group in the main chain mentioned in Japanese Patent Publication Nos. 55/734, Japanese Patent Laid-Open No. 50/054,672 and Japanese Patent Publication Nos. 59/14,735, 57/18,175, 57/18,176 and 57/56,059; and cationic pendant-type polymer having a cationic dissociating group in the side chain mentioned in Japanese Patent Publication Nos. 53/13,223, 57/15,376, 53/45,231, 55/145,783, 55/65,950, 55/67,746, 57/11,342, 57/19,735 and 58/56,858, Japanese Patent Laid-Open No. 61/027,853 and Japanese Patent Publication No. 62/9,346. Among them, preferred one is that where the conductive substance is in fine particles and they are finely dispersed in and added to the above nucleic acid-adsorptive solid carrier and, with regard to preferred conductive substance used therefor, it is preferred to contain conductive fine particles comprising metal oxide or compounded oxide thereof, ionene conductive polymer as mentioned in Japanese Patent Laid-Open No. 09/203,810 or quaternary ammonium cationic conductive polymer particles having an intermolecular cross-linking. Preferred particle size is within a range of 5 nm to 10 μm and more preferred range is dependent upon the type of the fine particles used. With regard to examples of metal oxide constituting the conductive fine particles, preferred ones are ZnO, TiO₂, SnO₂, Al₂O₃, In₂O₃, SiO₂, MgO, MoO₂, V₂O₅, etc. and compounded oxides thereof and ZnO, TiO₂ and SnO₂ are particularly preferred. With regard to examples where heteroatoms are contained, addition of Al, In, etc. to ZnO, addition of Nb, Ta, etc. to TiO₂ or addition of Sb, Nb, halogen element, etc. to SnO₂ is effective. Adding amount of such a heteroatom is preferred to be within a range of 0.01 to 25 mol % and the range of 0.1 to 15 mol % is particularly preferred. It is preferred that the nucleic acid-adsorptive solid carrier contains 0.01% to 20% in terms of volume fraction of such a conductive powder of metal oxide having a specific structure where bulk viscosity is not more than 107 Ωcm or, particularly, not more than 105 Ωcm, primary particle size is 100 Å to 0.2 Ωm and long diameter of higher-order structure is 30 nm to 6 μm. Characteristic of a cationic conductive polymer of a cross-linking type as a dispersible granular polymer is that, since cationic component in particles is able to be retained in high concentration and high density, it has excellent conductivity and, in addition, no deterioration of conductivity is noted even under low relative humidity and, in spite of the fact that particles are well dispersed in a dispersed state, adhesive force among particles is good during a film-formation process after flowing in the case of a form of membrane whereby membrane strength is high and resistance to chemicals is excellent. Dispersible granular polymer which is a cationic conductive polymer of a cross-linking type is usually within a particle size range of about 10 nm to 1,000 nm and, preferably, particle size within a range of 20 nm to 300 nm is used. The dispersible granular polymer used here has an appearance of transparent or a slightly turbid solution by naked eye observation but it is a polymer which is noted as a granular dispersion under an electron microscope. It is also possible to utilize an organic electron conductive organic compound. Its examples are polythiophene, polypyrrole, polyaniline, polyacetylene and polyphosphazene. They are preferably used as an acid donor in a complex with polystyrene sulfonic acid, perchloric acid, etc.

It is possible to add a deterioration preventer (such as antioxidant, peroxide degrading agent, radical inhibitor, metal inactivating agent, acid scavenger and amine) and an ultraviolet preventer to the above-mentioned nucleic acid-adsorptive solid carrier. With regard to such deterioration preventer and ultraviolet preventer, those which are mentioned in Japanese Patent Laid-Open Nos. 60/235,852, 03/199,201, 05/190,707, 05/194,789, 05/271,471, 06/107,854, 06/118,233, 06/148,430, 07/011,056, 07/011,055, 07/011,056, 08/029,619, 08/239,509, 07/011,056, 2000/204,173, 05/197,073, 05/194,789, 06/107,854, 60/235,852, 12/193,821, 08/029,619, 06/118,233, 06/148,430, 2002/265,636, 05/197,073, etc. may be preferably used. An example of the particularly preferred deterioration preventer is dibutylhydroxytoluene (BHT).

Adding amount thereof is preferably 0.01 to 1% by weight and, more preferably, 0.01 to 0.08% by weight of the solution (dope) to be prepared. When the adding amount is less than 0.01% by weight, effect of the deterioration preventer is rarely noted. When the adding amount is more than 1% by weight, there may be some cases where bleeding out (oozing) of the deterioration preventer onto the surface of the solid carrier is noted. Preferred deterioration preventer in the present invention is a liquid at 25° C. having a boiling point of 200° C. or higher or is a solid having a melting point of 25 to 250° C. More preferable deterioration preventer is a liquid at 25° C. having a boiling point of 250° C. or higher or is a solid having a melting point of 25 to 200° C. When the deterioration preventer is liquid, its purification is usually conducted by a vacuum distillation and the higher vacuum, the better. For example, 100 Pa or lower is preferred. It is also particularly preferred to purify using a vacuum distillation apparatus. When a plasticizer is solid, it is usual to conduct by recrystallization using a solvent, filtration, washing and drying.

A surfactant may be added to the above-mentioned nucleic acid-adsorptive solid carrier. With regard to the surfactant, that which is mentioned in Japanese Patent Laid-Open No. 2002/265,636, Japanese Patent Publication No. 55/031,418, “Tables of Surfactants, etc.” (2001) (Japan Surfactant Industry Association), “Applications of Surfactants” (by Takao Kaniyone, published by Saiwai Shobo on Sep. 1, 1980), etc. may be preferably used although they are non-limitative. For the preferred surfactant in the present invention, there is no particular limitation for its type and using amount but any amount may be used so far as an aimed surfactant is produced.

If necessary, the nucleic acid-adsorptive solid carrier may contain a releasing agent so that load for release upon manufacture is made little. As to the releasing agent as such, surfactant is effective and any of phosphate type, sulfonate type, carboxylate type, nonionic type, cationic type, etc. may be used without particular limitation. They are mentioned, for example, in Japanese Patent Laid-Open Nos. 61/243,837 and 2000/099,847. An acid or a salt thereof where acid dissociation index pKa is 1.93 to 4.50 [preferably 2.0 to 4.4, more preferably 2.2 to 4.3 (such as 2.5 to 4.0) and, particularly preferably, 2.6 to 4.3 (such as 2.6 to 4.0)] is preferred as a releasing agent. That may be any of inorganic acid and organic acid. The pKa of the acid may be referred to “Kagaku Binran (Handbook of Chemistry), Fundamental Part II, Revised Third Edition” edited by the Chemical Society of Japan and published by Maruzen. A releasing agent mentioned in Japanese Patent Laid-Open No. 2002/265,636 may be preferably used as well. In those descriptions, there are many preferred descriptions not only for releasing agents but also for utilization method or characteristics thereof and they may be also preferably used in the nucleic acid-adsorptive solid carrier of the present invention.

A coloring agent may be added to the above nucleic acid-adsorptive solid carrier. With regard to the coloring agent, organic, inorganic and organic-inorganic-compounded coloring agents such as coloring material, dye, pigment, oxidative coloring pigment, reductive coloring pigment, pH indicator, fluorescent pigment, coupling pigment, ultraviolet absorptive pigment, infrared absorptive pigment, near-infrared absorptive pigment, pressure-sensitive pigment, photochromic pigment, thermochromic pigment, electrochromic pigment, organic coloring pigment, pigment for food, organic nonlinear optical pigment, chemical luminescence pigment, pigment for pharmaceuticals, pigment for medical diagnosis, pigment for cosmetics, pigment for semiconductor laser, pigment for sublimation transcription, pigment for fusion transcription, heat-sensitive pigment, leuco pigment, electromagnetic absorptive pigment, photoconductive pigment and chargeable pigment which have been known already may be used either solely or jointly in a desired concentration or together with a dispersing agent such as surfactant or protective polymer in a desired concentration although they are non-limitative.

A reinforcing agent may be added to the above nucleic acid-adsorptive solid carrier in order to enhance the strength of a membrane. With regard to the reinforcing agent, examples of the preferably used ones are glass fiber, carbon fiber, silicon fiber, cellulose fiber, pulp fiber, potassium titanate fiber, silicon carbide whisker, silicon nitride whisker, zinc oxide whisker, aluminum borate whisker, magnesium basic sulfate, fibrous xonotlite, calcium titanate whisker, silicon carbide (SiC) whisker and whisker-shaped calcium carbonate although they are non-limitative but anything may be used so far as it is fibrous or is crystals in needles. It is also possible to add a synthetic polymer for enhancing a resistance against bending and, although polyurethane mentioned in Japanese Patent Laid-Open No. 54/011,081 may be preferably used, that is non-limitative.

A cross-linking agent may be added to the above nucleic acid-adsorptive solid carrier. With regard to the cross-linking agent, that which has been known already may be used and it is preferred to select an appropriate type depending upon a functional group of the solid carrier material. When a functional group is hydroxyl group, cross-linking agent mentioned in Japanese Patent Laid-Open Nos. 07/256,066, 03/058,431, etc. may be preferably used although they are non-limitative.

A moisturizing agent may be added to the nucleic acid-adsorptive solid carrier. With regard to the moisturizing agent, those which are mentioned in Japanese Patent Laid-Open No. 63/256,066, Japanese Patent Publication No. 03/068,431, etc. may be preferably used although they are non-limitative.

The nucleic acid-adsorbing porous membrane is capable of passing a solution through the inside of the membrane, and having a thickness of 10 to 500 μm is preferred. It is more preferred to have the thickness in a range of 50 to 250 μm. It is preferable to have thinner thickness in the reason for easier washing.

The nucleic acid-adsorbing porous membrane capable of passing a solution through the inside of the membrane having the minimum pore size of 0.22 μm or more is preferred. Having the minimum pore size of 0.5 μm or more is more preferred. In addition, using a porous membrane having the ratio of the maximum pore size and the minimum pore size to be 2 or more is preferred. As a result, sufficient surface area for adsorbing nucleic acid can be obtained, and the pores are not clogged easily. More preferable ratio of the maximum pore size and the minimum pore size is 5 or more.

The nucleic acid-adsorbing porous membrane capable of passing a solution through the inside of the membrane having the percentage of porosity in a range of 50 to 95% is preferred. More preferable percentage of porosity is in a range of 65 to 80%. Further, having a bubble point in a range of 0.1 to 10 kgf/cm² is preferred. More preferable bubble point is in a range of 0.2 to 4 kgf/cm².

The nucleic acid-adsorbing porous membrane capable of passing a solution through the inside of the membrane having a pressure loss in a range of 0.1 to 100 kPa is preferred. As a result, a uniformed pressure can be obtained at pressurized states. More preferable pressure loss is in a range of 0.5 to 50 kPa. Herein, the term “pressure loss” represents the minimum pressure necessary for passing water through per 100 μm thickness of a membrane.

The nucleic acid-adsorbing porous membrane capable of passing a solution through the inside of the membrane having an amount of water percolation, at the time of passing water through under 1 kg/cm² pressure at 25° C., in a range of 1 to 5000 mL per 1 cm² membrane for 1 minute is preferred. More preferable amount of water percolation, at the time of passing water through under 1 kg/cm² pressure at 25° C., is in a range of 5 to 1000 mL per 1 cm² membrane for 1 minute.

The nucleic acid-adsorbing porous membrane capable of passing a solution through the inside of the membrane having an amount of nucleic acid-adsorption of 0.1 μg or more per 1 mg of a porous membrane is preferred. More preferable amount of nucleic acid-adsorption is 0.9 μg or more per 1 mg of a porous membrane.

The nucleic acid-adsorbing porous membrane capable of passing a solution through the inside of the membrane having a cellulose derivative, which does not dissolve in less than 1 hour, but dissolves in less than 48 hours when a square porous membrane having a side length of 5 mm is deposited in 5 mL of trifluoroacetic acid is preferred. Further, a cellulose derivative, which dissolves in less than 1 hour when a square porous membrane having a side length of 5 mm is deposited in 5 mL of trifluoroacetic acid, but does not dissolve in less than 24 hours when deposited in 5 mL of dichloromethane is preferred. Among them, a cellulose derivative, which dissolves in less than 1 hour when a square porous membrane having a side length of 5 mm is deposited in 5 mL of trifluoroacetic acid, but does not dissolve in less than 24 hours when deposited in 5 mL of dichloromethane is more preferred.

When passing a nucleic acid mixture solution through a nucleic acid-adsorbing porous membrane, it is preferred that passing the nucleic acid mixture solution from one side to another side allows the solution to uniformly contact with the porous membrane. When passing a nucleic acid mixture solution through a nucleic acid-adsorbing porous membrane, it is preferred that passing the nucleic acid mixture solution through the nucleic acid-adsorbing porous membrane from a bigger pore size to a smaller pore size in the purpose of not clogging the pore easily.

When passing a nucleic acid mixture solution through a nucleic acid-adsorbing porous membrane, it is preferred to have the flow rate in a range of 2 to 1500 μL/sec per unit area cm² of the membrane to obtain suitable contact time of the solution to the porous membrane. When the contact time of the solution to the porous membrane is too short, sufficient separation and purification effect cannot be obtained, and when too long, it is not preferred due to its operability. The flow rate in a range of 5 to 700μL/sec per unit area cm² of the membrane is preferred.

In addition, the nucleic acid-adsorbing porous membrane capable of passing a solution through the inside of the membrane can be used in one layer, but also can be used in multi-layers. The multi-layers of the nucleic acid-adsorbing porous membrane can be identical to or different from each other.

A nucleic acid-separating cartridge receiving a nucleic acid-adsorptive porous membrane through which the above-mentioned solution is able to pass in a container having at least two openings is able to be preferably used. Further, a nucleic acid-separating cartridge receiving plural nucleic acid-adsorptive porous membranes through which the above-mentioned solution is able to pass in a container having at least two openings is able to be preferably used. In that case, the plural nucleic acid-adsorptive porous membranes received in the container having at least two openings may be same or different.

The plural nucleic acid-adsorptive porous membranes may be a combination of a nucleic acid-adsorptive porous membrane of an inorganic material with a nucleic acid-adsorptive porous membrane of an organic material. An example thereof is a combination of glass filter with a porous membrane of regenerated cellulose. The plural nucleic acid-adsorptive porous membrane may also be a combination of a nucleic acid-adsorptive porous membrane of an inorganic material with a nucleic acid-nonadsorptive porous membrane of an organic material. An example thereof is a combination of glass filter with a porous membrane of Nylon or polysulfone.

The above-illustrated nucleic acid-adsorptive porous membrane may be made into a form which is other than membrane depending upon the shape of the cartridge. For example, it may be made into a form of chip or block.

The cartridge for separation and purification of nucleic acid should not comprise other members except for comprising a container having at least two openings wherein the cartridge for separation and purification of nucleic acid receives the nucleic acid-adsorbing porous membrane, which solutions can pass through as mentioned above, in an inside of the container. Examples of materials for the container include plastics such as polypropylene, polystyrene, polycarbonate and polyvinyl chloride can be used. In addition, a biodegradable material can also be used preferably. Further, the container can be transparent or colored.

The cartridge for separation and purification of nucleic acid comprising the means for distinguishing between each cartridge for separation and purification of nucleic acid can be used. The means for distinguishing between each cartridge for separation and purification of nucleic acid may include a bar code, a 2-dimensional bar code, a magnetic tape, and an IC card.

It is also possible to use a cartridge for separation and purification of nucleic acid having in such a structure that a nucleic acid-adsorptive membrane is able to be easily taken out from a container having at least two openings.

A test sample to be used in the invention is not limited as long as a test sample contains nucleic acid, for examples thereof in the field of diagnostics include body fluids collected as test samples, such as whole blood, plasma, serum, urine, faeces, semen and saliva, or plants (or a part thereof), animals (or a part thereof), bacteria, virus, cultured cells, solutions prepared from biological materials such as lysates and homogenates of the above samples.

These test samples are treated with an aqueous solution comprising a reagent which dissolves cell membranes and nuclear membrane, and solubilizes nucleic acid, so called a nucleic acid-solubilizing reagent. This enables cell membranes and nuclear membranes to be dissolved, and enables nucleic acid to be dispensed into the aqueous solution to obtain a mixture solution of nucleic acid.

The nucleic acid-containing sample may be a sample containing a single nucleic acid, or may be a sample containing different, plural kinds of nucleic acids. Nucleic acids to be recovered are not limited as to kind, and may be DNA or RNA, single-stranded chain or double-stranded chain, and straight or cyclic. The number of samples may be one or plural (parallel treatment of plural samples using plural vessels). The length of nucleic acid to be recovered is not particularly limited, either, and a nucleic acid of any length between, for example, from several bp to several Mbp can be used. In view of handling convenience, the length of a nucleic acid to be recovered is generally from about several bp to about several hundreds kbp. The method of the invention for separation and purification of nucleic acid enables one to recover a comparatively longer nucleic acid expeditiously than that obtained by the conventional simple method for separation and purification of nucleic acid, and can be employed for recovering a nucleic acid of preferably 50 kbp or more, more preferably 70 kbp or more, still more preferably 100 kbp or more. In view of recovering a longer DNA, it is preferable to conduct stirring and pipetting mildly.

A step of obtaining a sample solution containing nucleic acids from a sample by lysis of cell membrane and nuclear membrane to thereby solubilize nucleic acids is described below. In the invention, a nucleic acid-solubilizing reagent is used for solubilizing nucleic acid by lysis of cell membrane and nuclear membrane. Examples of the nucleic acid-solubilizing reagent include solutions containing compounds selected from a chaotropic salt, a surfactant, a defoaming agent, a protease and a nucleic acid stabilizing agent.

As a method for obtaining a sample solution containing nucleic acids from a sample by lysis of cell membrane and nuclear membrane to thereby solubilize nucleic acids, there is illustrated a method including the steps of:

(I) injecting a sample containing cells or viruses into a container;

(II) adding a nucleic acid-solubilizing reagent solution containing a chaotropic salt or a surfactant to the container, and mixing the sample with the nucleic acid-solubilizing reagent solution;

(III) incubating the resultant mixed solution; and

(IV) adding a water-soluble organic solvent to the incubated mixed solution.

In the step of obtaining a sample solution containing nucleic acids from a sample by lysis of cell membrane and nuclear membrane to thereby solubilize nucleic acids, adaptability for automated processing is improved by subjecting the sample to homogenizing treatment. Such homogenizing treatment can be conducted, for example, by ultrasonic wave treatment, treatment using a sharp projection, high-speed stirring treatment, treatment of extruding through fine pores or treatment using glass beads.

Also, in the step of obtaining a sample solution containing nucleic acids from a sample by lysis of cell membrane and nuclear membrane to thereby solubilize nucleic acids, the recovering amount and recovering yield of nucleic acid can be improved by using a nucleic acid-solubilizing reagent containing a protease, thus reduction of the necessary amount of a sample containing nucleic acids and acceleration of the analysis becoming possible.

As such protease, at least one protease selected from among serine protease, cysteine protease, metal protease, etc. can preferably be used. Also, a mixture of plural kinds of proteases may preferably be used.

Serine protease is not particularly limited and, for example, protease K can preferably be used. Cysteine protease is not particularly limited and, for example, papain and cathepsin may preferably be used.

Metal protease is not particularly limited and, for example, carboxypeptidase may preferably be used.

The protease can be used, upon addition, in an amount of preferably from 0.001 IU to 10 IU, more preferably from 0.01 IU to 1 IU, per ml of the whole reaction system.

Also, as the protease, a protease not containing nuclease can preferably be used. Also, a protease containing a stabilizing agent can preferably be used. As the stabilizing agent, a metal ion can preferably be used. Specifically, magnesium ion is preferable, and can be added in the form of, for example, magnesium chloride. Incorporation of a stabilizing agent for a protease enables one to reduce the amount of protease necessary for recovery of nucleic acids to a slight amount, which serves to reduce the cost required for recovery of nucleic acids. The amount of the stabilizing agent for protease is preferably from 1 to 1000 mmol/L, more preferably from 10 to 100 mmol/L, based on the whole amount of the reaction system.

The protease may be used as one reagent obtained by previously mixing with other reagents such as a chaotropic salt and a surfactant, thus being used for recovery of nucleic acids.

Alternatively, the protease may be used as a separate reagent from other reagents such as a chaotropic salt and a surfactant.

In the latter case, a sample is first mixed with a reagent containing a protease, and the mixture is then mixed with a reagent containing a chaotropic salt and a surfactant. Or, the protease may be mixed after first mixing a sample with the reagent containing a chaotropic acid and a surfactant.

Also, it is possible to dropwise add from a container retaining a protease directly like an eye lotion to a sample or a mixture of a sample and a reagent containing a chaotropic salt and a surfactant. In this case, operation can be simplified.

The nucleic acid-solubilizing reagent may preferably be fed in a dry state as well. Also, a container previously containing a protease in a dried state, for example, by freeze-drying can be used. It is also possible to obtain a sample solution containing nucleic acid by using both the nucleic acid-solubilizing reagent to be fed in a dry state and a container previously containing a dried protease.

The concentration of a chaotropic salt in the nucleic acid-solubilizing reagent is preferably 0.5 mol/L or more, more preferably from 0.5 to 4 mol/L and even more preferably from 1 to 3 mol/L. As the chaotropic salt, known chaotropic salts can be used without any particular limitations. For example, guanidine salt, sodium isothiocyanate, sodium iodide and potassium iodide can be used. Especially, guanidine salt is preferred. Examples of guanidine salt include guanidine hydrochloride, guanidine isothiocyanate and guanidine thiocyanate salt (guanidine thiocyanate), and especially guanidine hydrochloride or guanidine thiocyanate salt is preferred. These salts can be used alone or in combinations of two or more.

It is possible to use a chaotropic substance such as urea instead of a chaotropic salt.

Surfactants, for example, include a nonionic surfactant, a cationic surfactant, an anionic surfactant, an amphoteric surfactant.

In the invention, the nonionic surfactant and the cationic surfactant can be preferably used.

Nonionic surfactants include a polyoxyethylene alkyl phenyl ether-based surfactant, a polyoxyethylene alkyl ether-based surfactant, and fatty acid alkanolamide, and the preferable one is a polyoxyethylene alkyl ether-based surfactant. Among the polyoxyethylene (POE) alkyl ether surfactant, POE decyl ether, POE lauryl ether, POE tridecyl ether, POE alkylenedecyl ether, POE sorbitan monolaurate, POE sorbitan monooleate, POE sorbitan monostearate, tetraoleic polyoxyethylene sorbit, POE alkyl amine, and POE acetylene glycol are more preferred.

Cationic surfactants include cetyl trimethyl ammonium bromide, dodecyl trimethyl ammonium chloride, tetradecyl trimethyl ammonium chloride, cetyl pyridinium chloride.

These surfactants can be used alone or in combinations of two or more. The concentration of the surfactant in the nucleic acid-solubilizing reagent is preferably from 0.1 to 20% by weight.

When nucleic acid other than RNA such as DNA is recovered, it is preferred to add an RNA-degrading enzyme to a solution of a solubilizing reagent for nucleic acid in a step where nucleic acid is made soluble and a sample solution containing nucleic acid is prepared from a test body. In that case, interference by RNA coexisting with the recovered nucleic acid is able to be reduced. It is also preferred to add an inhibitor for a DNA degrading enzyme. On the contrary, in the case of recovery of nucleic acid other than DNA such as RNA, it is preferred to add a DNA degrading enzyme to a solution for a solubilizing reagent for nucleic acid. In that case, interference by DNA coexisting with the recovered nucleic acid is able to be reduced. It is also preferred to add an inhibitor for a RNA degrading enzyme. With regard to an inhibitor for RNA degrading enzyme, that which specifically inhibits the RNA degrading enzyme is preferred. There is no particular limitation for the RNA degrading enzyme and an enzyme which specifically degrades RNA such as ribonuclease H(RNase H) may be preferably used. There is no particular limitation for the DNA degrading enzyme and an enzyme which specifically degrades DNA such as DNase I may be preferably used. Nucleic acid degrading enzyme and inhibitor for nucleic acid degrading enzyme may be used in commonly used concentrations. It is also possible to subject them to a common warming treatment. It is preferred that the warming treatment is conducted together with a treatment with protein-degrading enzyme.

As the nucleic acid stabilizing agent, one having a reaction to inactivate a nuclease activity can be exemplified. Depending on a test sample, there are cases where nuclease, which degrades nucleic acid, is comprised thereto so that when nucleic acid is homogenized, nuclease reacts with nucleic acid, so as to result in a remarkable reduction of a yield amount. For the purpose of avoiding this, a stabilizing agent having a function to inactivate nuclease can be coexisted in a nucleic acid-solubilizing solution. As a result, improvements in a recovering yield and a recovering efficiency of nucleic acid lead to the minimization and acceleration of a test sample.

As the nucleic acid stabilizing agent having functions to inactivate the nuclease activity, a compound used routinely as a reducing agent can be used. Examples of reducing agents include hydrogenated compounds such as a hydrogen atom, hydrogen iodide, hydrogen sulfide, aluminum lithium hydride, and sodium borohydride; a highly electropositive metal such as alkaline metal, magnesium, calcium, aluminum, and zinc, or their amalgam; organic oxides such as aldehyde-based, sugar-based, formic acid, and oxalic acid; and mercapto compounds. Among these, the mercapto compounds are preferable. Examples of mercapto compounds include N-acetyl cysteine, mercapto ethanol, and alkyl mercaptane or the like. The mercapto compounds can be used alone or in combinations of two or more.

The concentration of the nucleic acid stabilizing agent in the nucleic acid-solubilizing reagent is preferably from 0.1 to 20% by weight, and more preferably from 0.3 to 15% by weight. The concentration of the mercapto compounds in the nucleic acid-solubilizing reagent is preferably from 0.1 to 10% by weight, and more preferably from 0.5 to 5% by weight.

In the above-mentioned step where cell membrane and nuclear membrane are dissolved, nucleic acid is made soluble and a sample solution containing nucleic acid is prepared from a test body, it is preferred that an antifoaming agent (defoaming agent) is contained in a sample solution containing nucleic acid.

As the defoaming agent, a silicon-based defoaming agent (e.g., silicon oil, dimethyl polysiloxane, silicon emersion, denatured polysiloxane, silicon compound, etc.), alcohol-based defoaming agent (e.g., acetylene glycol, heptanol, ethyl exanol, superhigh grade alcohol, polyoxy alkylene glycol, etc.), ether-based defoaming agent (e.g., heptyl cellosolve, nonyl cellosolve-3-heptylcorbitol, etc.), fatty oil-based defoaming agent (e.g., animal and plant fat, etc.), fatty acid-based defoaming agent (e.g., stearic acid, oleic acid, palmitic acid, etc.), metallic soap-based defoaming agent (e.g., aluminum stearate, calcium stearate, etc.), fatty acid ester-based defoaming agent (e.g., a natural wax, tributyl phosphate, etc.), phosphate ester-based defoaming agent (e.g., sodium octyl phosphate, etc.), amine-based defoaming agent (e.g., diamyl amine, etc.), amide-based defoaming agent (e.g., amide stearate, etc.), and other defoaming agents (e.g., ferric sulfate, bauxite, etc.) can be exemplified. These defoaming agent can be used alone or in combinations of two or more. Two compounds combined from silicon-based and alcohol-based defoaming agents are especially preferred.

The concentration of a defoaming agent in nucleic acid-solubilizing reagent is preferably in a range of 0.1 to 10% by weight.

The nucleic acid-solubilizing reagent is preferred to be supplied in a drying state. Further, it is possible to use a container preliminary contains a protease in a drying state such as a lyophilization. The test sample containing nucleic acid can also be obtained by using both of above-mentioned the nucleic acid-solubilizing reagent in the drying state and the container preliminary contains the protease in the drying state. When obtaining the test sample containing nucleic acid in above-mentioned method, the stage stability of the nucleic acid-solubilizing reagent and the protease is good, and it is possible to simplify the operations without changing the yields of separated and purified nucleic acids.

The method for mixing a sample and the nucleic acid-solubilizing reagent solution is not particularly limited.

Upon mixing, it is preferable to mix at 30 to 3000 rpm for 3 minutes using a stirrer, whereby the yield of nucleic acid separated and purified can be increased. Also, it is also preferable to mix by conducting end-over-end mixing 5 to 30 times. Also, mixing can be conducted by repeating pipetting operation 10 to 50 times. In this case, the yields of separated and purified nucleic acids can be increased by simple operation.

The yields of separated and purified nucleic acids can be increased by incubating the mixture of a sample and a nucleic acid-solubilizing reagent solution at an optimal temperature for a protease for an optimal reaction time. The incubation temperature is usually from 20° C. to 70° C., preferably an optimal temperature for the protease, and the incubation time is usually from 1 minute to 18 hours, preferably an optimal incubation time for the protease. The incubation method is not particularly limited, and can be conducted by dipping into a warm bath or a heating chamber.

In the above-mentioned step for producing a sample solution containing nucleic acid from a test body where the above-mentioned cell membrane and nuclear membrane are dissolved and nucleic acid is made soluble, examples of a water-soluble organic solvent which is added to an incubated mixed solution are alcohols, acetone, acetonitrile and dimethylformamide. Alcohols are able to be used particularly preferably. Any of primary, secondary and tertiary alcohols may be used as an alcohol. Alcohols which are methyl alcohol, ethyl alcohol, propyl alcohol and an isomer thereof and butyl alcohol and isomers thereof may be preferably used. Ethyl alcohol may be used more preferably. Each of those water-soluble organic solvents may be used solely or plural thereof may be used in combination. Concentration of such a water-soluble organic solvent in a solution of a solubilizing reagent for nucleic acid is preferred to be 1 to 20% by weight. Final concentration of such a water-soluble organic solvent in a sample solution containing nucleic acid is preferred to be 5 to 90% by weight.

In the step of obtaining a sample solution containing nucleic acids from a sample by lysis of cell membrane and nuclear membrane to thereby solubilize nucleic acids, the nucleic acid-solubilizing reagent solution has a pH of preferably 5 to 10, more preferably 6 to 9, still more preferably 7 to 8.

In the above-mentioned step for producing a sample solution containing nucleic acid from a test body where the above-mentioned cell membrane and nuclear membrane are dissolved and nucleic acid is made soluble, surface tension of the resulting sample solution containing nucleic acid is preferred to be 0.05 J/m² or less, viscosity thereof is preferred to be 1 to 10,000 mPa and specific gravity thereof is preferred to be 0.8 to 1.2

An illustration will now be made for a washing step as hereinafter. As a result of conducting a washing, recovered amount and purity of nucleic acid are enhanced and necessary amount of a test body containing nucleic acid is able to be made small. With regard to the washing step, one step will be acceptable for a purpose of quickening while, if purity is more important, it is preferred to repeat the washing for plural times.

In the washing step, a washing solution is provided to a cartridge for separation and purification of nucleic acid receiving the nucleic acid-adsorbing porous membrane by using a tube, a pipette, an automatic injection apparatus, or a providing means having the like function. The washing solution is provided from one opening of the cartridge for separation and purification of nucleic acid (the one opening where a nucleic acid mixture solution containing nucleic acid is injected), and a pressure difference-generating apparatus connecting to the one opening (e.g., a dropper, a syringe, a pump, a power pipette etc.) is used. Thereby making an inside of the cartridge for separation and purification of nucleic acid into a pressurized state, so as to pass the washing solution through the nucleic acid-adsorbing porous membrane, and discharge the washing solution from another opening different from the one opening. Additionally, the washing solution can be provided into one opening and discharged from the same opening. Further, the washing solution can be provided to another opening different from the one opening which the nucleic acid mixture solution containing nucleic acid is provided to, and discharged from the same opening. Among them, providing into one opening of the cartridge for separation and purification of nucleic acid, passing through nucleic acid-adsorbing porous membrane and discharging from another opening different from the one opening is much preferred due to its excellent washing efficiency.

In a washing procedure, the amount of a washing solution is preferably 2 μl/mm² or more. When large quantity of the washing solution is used, the washing effect could improve, but in order to maintain the operationability and prohibit the sample from discharging, 200 μl/mm² or less is preferred.

When a washing solution is supplied in a washing step and then allowed to stand for 50 seconds or longer, a recovered amount of nucleic acid is significantly improved. When more time is spent therefor, stabilization in the recovered amount can be expected but, in view of a quick operation, 1,000 seconds or shorter is selected.

In the present invention, time after a washing solution is supplied by an automatic extracting machine is counted whereby the time for being allowed to stand is automatically controlled.

In a washing procedure, when passing a washing solution through a nucleic acid-adsorbing porous membrane, it is preferred to have the flow rate in a range of 2 to 1500 μl/sec per unit area (cm²) of the membrane, and more preferably in a range of 5 to 700 μl/sec. Normally, the passing speed is reduced to elongate the time so that washing is sufficiently conducted. However, preferably, by using the aforementioned range in the invention the step for separating and purifying RNA can be conducted rapidly without reducing the washing efficiency.

In the washing step, a temperature of the washing solution in a range of 4 to 70° C. is preferred. Further, a temperature of the washing solution at room temperature is more preferred. In addition to the washing step, stirring using an ultrasonic or a mechanical vibration can be applied to the cartridge for separation and purification of nucleic acid at the same time. On the other hand, washing can be done by conducting a centrifugation.

Generally, in a washing step, although an enzyme such as nucleic acid-degrading enzyme is not contained in a washing solution, an enzyme which degrades contaminants such as protein may be contained therein. In some cases, DNA-degrading enzyme, RNA-degrading enzyme, etc. may be also contained therein. As a result of use of a washing solution containing a DNA-degrading enzyme, only RNA in a test body is able to be recovered selectively. On the contrary, as a result of use of a washing solution containing an RNA-degrading enzyme, only DNA in a test body is able to be recovered selectively.

In the washing step, the washing solution is a solution containing at least one of water-soluble organic solutions and water-soluble salts is preferred. It is necessary for a washing solution to have ability that works to wash out impurities of the nucleic acid mixture solution, which are adsorbed onto the nucleic acid-adsorbing porous membrane along with nucleic acid. In this regard, the washing solution must have such a composition that it desorbs only impurities from the nucleic acid-adsorbing porous membrane, and not the nucleic acid. In the purpose, nucleic acid are very insoluble to water-soluble organic solvents such as alcohol, therefore the water-soluble organic solvent is suitable for desorbing other substances by maintaining nucleic acid. In addition, adding water-soluble salts enables to increase an adsorption effect of nucleic acid, thereby improving the selectively removing operation for impurities and unnecessary substances.

With regard to a water-soluble organic solvent to be contained in a washing solution, methanol, ethanol, isopropanol, n-propanol, butanol, acetone, etc. may be used and, among them, it is preferred to use ethanol. Amount of the water-soluble organic solvent contained in the washing solution is preferably 20 to 100% by weight and, more preferably, 40 to 80% by weight.

On the other hand, for the water-soluble salt contained in a washing solution, a halide salt is preferred and among them, a chloride salt is more preferred. Further, the water-soluble salt is preferably a monovalent or divalent cation, particularly an alkali metal and an alkali earth metal is preferred. And among them, a sodium salt and a potassium salt are most preferred. When the water-soluble salt is contained in the washing solution, the concentration thereof is preferable 10 mmol/L or more, and the upper limit is not particularly limited as long as the upper limit does not affect solubility of the impurities, 1 mol/L or less is preferred and 0.1 mol/L or less is more preferred. Above all, that the water-soluble salt is sodium chloride and sodium chloride is contained in 20 mmol/L or more is particularly preferred.

In addition, the washing solution is characterized in that a chaotropic substance is not contained therein. As a result, a possibility of having the chaotropic substance incorporated into a recovery step after the washing step can be reduced. In the recovery step, where the chaotropic substance is incorporated thereinto, it sometimes hinders an enzyme reaction such a PCR reaction or the like, therefore considering the afterward enzyme reaction, not including the chaotropic substance to a washing solution is ideal. Further, the chaotropic substance is corrosive and harmful, in this regard, it is extremely advantageous from an operational safety standpoint for the researcher not to use the chaotropic substance when unnecessary.

Herein, the chaotropic substance represents aforementioned urea, guanidine chloride, guanidine isothiocyanate, guanidine thiocyanate, sodium isothiocyanate, sodium iodide, potassium iodide, etc.

Since the washing solution has high wettability for a cartridge or the like container, the washing solution sometimes remains in the container during the washing step in the nucleic acid separation purification process, so that the recovery step after the washing step is contaminated with the washing solution to cause reduction of the purity of nucleic acid and reduction of the reactivity in the subsequent step. Thus, in the first method and the second method of the present invention, when adsorption and desorption of nucleic acid are carried out using a cartridge or the like container, it is important that a solution to be used in the adsorption or washing, particularly the washing solution, does not remain in the cartridge so that it does not exert influence upon the next step.

Accordingly, in order to prevent contamination of the recovering solution of the subsequent step with the washing solution of the washing step and thereby to keep residue of the washing solution in the cartridge to the minimum, it is desirable that surface tension of the washing solution is less than 0.035 J/m². When the surface tension is low, wettability of the washing solution for the cartridge is improved and volume of the residual solution can be controlled.

However, the ratio of water can be increased in order to increase the washing efficiency, but in that case, surface tension of the washing solution is increased and amount of the residual solution is increased. When surface tension of the washing solution is 0.035 J/m² or more, amount of the residual solution can be controlled by increasing water repellency of the cartridge. By increasing water repellency of the cartridge, droplets are formed, and amount of the residual solution can be controlled by flow down of the droplets. Examples of the method for increasing water repellency include coating of a water repellant such as silicon on the cartridge surface, kneading of a water repellant such as silicon at the time of the cartridge forming, and the like, though not limited thereto.

When the nucleic acid-adsorptive porous membrane according to the present invention is utilized, it is now possible to simplify the washing step. Thus, (1) frequency of passing of the washing solution through a nucleic acid-adsorptive porous membrane is now only once; (2) the washing step is able to be conducted at room temperature; (3) after the washing, the recovering solution is now able to be directly infused into a cartridge; and (4) any of or two or more of the above (1), (2) and (3) is possible. The reason is that, in the conventional method, the organic solvent contained in the washing solution is to be quickly removed and, therefore, a drying step is often necessary but, since the nucleic acid-adsorptive membrane according to the present invention is a thin membrane, the step is able to be omitted.

In the conventional step for separation and purification of nucleic acid, there is a problem that, during a washing step, a washing solution often splashes and adheres to other things whereby contamination (pollution) of the sample happens. Such a kind of contamination in a washing step is able to be suppressed by improvements in shapes of a waste solution container and a cartridge for separation and purification of nucleic acid receiving a nucleic acid-adsorptive membrane in a container having two openings.

As hereunder, a step where nucleic acid is desorbed from a nucleic acid-absorptive porous membrane and is recovered will be illustrated. In a recovering step, a recovering solution is supplied to a cartridge for separation and purification of nucleic acid equipped with a nucleic acid-adsorptive porous membrane using an automated infusion apparatus or a supplying means having the same function. A recovering solution is supplied from one of the openings (an opening wherefrom a sample solution containing nucleic acid is infused) of a cartridge for separation and purification of nucleic acid, passed through a nucleic acid-adsorptive porous membrane under such a state that inner area of the cartridge for separation and purification of nucleic acid is made vacuum using an apparatus for generation of difference in pressure connected to the opening and able to be discharged from an opening which is other than said one of the openings. It is also possible that a recovering solution is able to be supplied from said one of the openings and discharged from the same said one of the openings. It is further possible that a recovering solution is supplied from an opening which is other than said one of the openings of a cartridge for separation and purification of nucleic acid wherefrom a sample solution containing nucleic acid is supplied and also discharged therefrom. However, a method where a recovering solution is supplied from an opening of a cartridge for separation and purification of nucleic acid, passed through a nucleic acid-adsorptive porous membrane and discharged from an opening which is other than said one of the openings is excellent in a recovering efficiency and is more preferred.

When a recovering solution is supplied in a recovering step and then allowed to stand for 20 seconds or longer, a recovered amount of nucleic acid is significantly improved. When more time is spent therefor, stabilization in the recovered amount can be expected but, in view of a quick operation, 300 seconds or shorter is selected.

In the present invention, time after a recovering solution is supplied by an automatic extracting machine is counted whereby the time for being allowed to stand is automatically controlled.

Volume of a recovering solution to volume of the sample solution containing nucleic acid prepared from a test body is adjusted whereby desorption of nucleic acid is able to be carried out. Volume of the recovered solution containing nucleic acid which is separated and purified is dependent upon the amount of the test body used at that time. Although the commonly used amount of the recovered solution is from several tens to several hundred μl, that may be changed within a range of 1 μl to several tens ml when the sample amount is very little or, reversely, a large amount of nucleic acid is to be separated and purified.

For the recovering solution, purified distilled water, Tris/EDTA buffer and the like can preferably be used. Further, when providing the recovered nucleic acid to PCR (Polymerase Chain Reaction), the buffer solution (e.g., an aqueous solution having the final concentration of 50 mmol/L of KCl, 10 mmol/L of Tris-HCl, 1.5 mmol/L of MgCl₂) for PCR can be used.

It is preferred that pH of a recovering solution is 2 to 11 and, more preferably, 5 to 9. In addition, ionic strength and salt concentration particularly affect the elution of adsorbed nucleic acid. Preferably, the recovering solution has an ionic strength of 290 mmol/L or less and has a salt concentration of 90 mmol/L or less. As a result thereof, recovering rate of nucleic acid increases and much more nucleic acid is able to be recovered. The recovered nucleic acid may be either single-stranded or double-stranded.

When volume of a recovering solution is made small as compared with the initial volume of a sample solution containing nucleic acid, it is now possible to prepare a recovered solution containing concentrated nucleic acid. Preferably, the ratio of (volume of recovering solution):(volume of sample solution) is able to be made 1:100 to 99:100 and, more preferably, it is able to be made 1:10 to 9:10. As a result thereof, nucleic acid is now able to be easily concentrated without conducting an operation for concentrating in a step after separation and purification of nucleic acid. According to such a method, a method for producing a nucleic acid solution in which nucleic acid is concentrated as compared with a test body is able to be provided.

Another method is that desorption of nucleic acid is conducted under a condition where volume of a recovering solution is more than the initial volume of a sample solution containing nucleic acid whereby it is possible to prepare a recovering solution containing nucleic acid of a desired concentration and to prepare a recovering solution containing nucleic acid which is suitable for the next step (such as PCR). Preferably, the ratio of (volume of recovering solution):(volume of sample solution) is able to be made 1:1 to 50:1 and, more preferably, it is able to be made 1:1 to 5:1. As a result thereof, there is an advantage that, after separation and purification of nucleic acid, troublesomeness for adjustment of concentration is no longer necessary. In addition, as a result of use of a sufficient amount of a recovering solution, an increase in a recovering rate of nucleic acid from the porous membrane is able to be achieved.

There is no limitation for the infusing times for a recovering solution and that may be either once or plural times. Usually, when nucleic acid is to be separated and purified quickly and simply, that is carried out by means of one recovery while, when a large amount of nucleic acid is to be recovered, recovering solution may be infused for several times.

In a recovering step, it is possible that a recovering solution for nucleic acid is made in such a composition that is able to be used in the steps after that. Nucleic acid which is separated and purified is often amplified by a PCR (polymerase chain reaction) method. In that case, it is necessary that the separated and purified nucleic acid solution is diluted with a buffer which is suitable for a PCR method. When a buffer solution suitable for a PCR method is used for a recovering solution in a recovering step according to the present method, it is now possible to transfer to the next PCT step easily and quickly.

Also, in the recovering step, it is possible to add a stabilizing agent for preventing degradation of RNA recovered in the recovering solution of RNA. As the stabilizing agent, an antibacterial agent, a fungicide, a nucleic acid degradation inhibitor and the like can be added. As the nuclease inhibitor, EDTA and the like can be cited. In addition, as another embodiment, a stabilizer can also be added to the recovery container in advance.

Also, the recovery container to be used in the recovery step is not particularly limited, a recovery container prepared from a raw material having no absorption at 260 nm can be used. In that case, concentration of the recovered RNA solution can be measured without transferring it into other container. As the raw material having no absorption at 260 nm, quartz glass and the like can for example be used, though not limited thereto.

Nucleic acid is able to be separated and purified according to the following steps using a cartridge for separation and purification of nucleic acid receiving a nucleic acid-adsorptive porous membrane through which each solution is able to pass as mentioned already. Thus, (a) a step where a sample solution containing nucleic acid is infused into one of openings of a cartridge for separation and purification of nucleic acid receiving a nucleic acid-adsorptive porous membrane through which a solution is able to pass in a container having at least two openings, (b) a step where inner part of the cartridge for separation and purification of nucleic acid is made into a pressurized state using an apparatus for generation of pressure difference connected to said one of the openings of the cartridge for separation and purification of nucleic acid and the infused sample solution containing nucleic acid is passed through a nucleic acid-adsorptive porous membrane and discharged from another opening of the cartridge for separation and purification of nucleic acid whereby nucleic acid is adsorbed with the nucleic acid-adsorptive porous membrane, (c) a step where a washing solution is infused into said one of the openings of the cartridge for separation and purification of nucleic acid, (d) a step where inner part of the cartridge for separation and purification of nucleic acid is made into a pressurized state using an apparatus for generation of pressure difference connected to said one of the openings of the cartridge for separation and purification of nucleic acid and the infused washing solution is passed through a nucleic acid-adsorptive porous membrane and discharged from another opening whereby the nucleic acid-adsorptive porous membrane is washed under such a state where nucleic acid is still adsorbed, (e) a recovering solution is infused into said one of the openings of the cartridge for separation and purification of nucleic acid and (f) a step where inner part of the cartridge for separation and purification of nucleic acid is made into a pressurized state using an apparatus for generation of pressure difference connected to said one of the openings of the cartridge for separation and purification of nucleic acid and the infused recovering solution is passed through a nucleic acid-adsorptive porous membrane and discharged from another opening whereby nucleic acid is desorbed from the nucleic acid-adsorptive porous membrane and discharged to outside of the cartridge for separation and purification of nucleic acid may be listed.

In each of the above-mentioned steps (b), (d) and (f), nucleic acid-containing sample solution, washing solution or recovering solution is passed through a nucleic acid-adsorptive porous membrane under a pressurized state. More preferably, in each of the above-mentioned steps (b), (d) and (f), nucleic acid-containing sample solution, washing solution or recovering solution is infused into one of the openings of a cartridge for separation and purification of nucleic acid receiving said nucleic acid-adsorptive porous membrane in a container having at least two openings and inner part of the cartridge is made into a pressurized state using an apparatus for generation of pressure difference connected to said one of the openings of the cartridge whereby each of said infused solutions is passed therethrough and discharged from another opening. As a result of passing the nucleic acid-containing sample solution, washing solution or recovering solution is passed through the above-mentioned porous membrane in a pressurized state, the apparatus is able to be automated in a compact manner and that is preferred. Pressurization is conducted preferably to an extent of about 10 to 200 kpa or, more preferably, 40 to 100 kpa.

In the above-mentioned step for separation and purification of nucleic acid, it is possible that the step from the initial infusion of sample solution containing nucleic acid until preparation of nucleic acid outside the cartridge for separation and purification of nucleic acid finishes within 10 minutes or, under a suitable circumstance, within 2 minutes. It is also possible in the above-mentioned step after separation and purification of nucleic acid that nucleic acid is prepared in a yield of 50% by weight or more and, under a suitable circumstance, 90% by weight or more to the total amount contained in the test body.

In the above-mentioned step for separation and purification of nucleic acid, it is possible to recover nucleic acid having a molecular range of as broad as 1 kpb to 200 kpb and, particularly, 20 kpb to 140 kpb. Thus, as compared with the conventionally conducted spin column method using glass filter, nucleic acid of longer chain is able to be recovered.

In addition, in the aforementioned steps of separation and purification of nucleic acid, nucleic acid in case of including DNA having a purity corresponding to an absorbance measurement of ultraviolet-visible spectrophotometer (260 nm/280 nm) of 1.6 to 2.0 and in case of including RNA having a purity corresponding to an absorbance measurement of ultraviolet-visible spectrophotometer (260 nm/280 nm) of 1.8 to 2.2 can be recovered, and nucleic acid of high purity with little amount of impurities contamination can be obtained for constant. Further, those having an absorbance measurement of ultraviolet-visible spectrophotometer (260 nm/280 nm) of around 1.8 for DNA and around 2.0 for RNA can be recovered.

### EXAMPLE

### Example 1

As hereinafter, a measuring test of yield of nucleic acid collected by conducting a nucleic acid extracting step using the method for extracting nucleic acid according to the present invention was conducted. In the test, an apparatus for extraction of nucleic acid which was illustrated in the above-mentioned embodiments was used.

(1) Preparation of a Container for Separation and Purification of Nucleic Acid

As to a container for separation and purification of nucleic acid used as a cartridge in this Example, a container for separation of nucleic acid having 7 mm inner diameter, receiving a solid phase for adsorption of nucleic acid and having two openings was prepared using high-impact polystyrene. Diameter of the lower opening was made 2.5 mm.

(2) Unit for Separation and Purification of Nucleic Acid

As to a nucleic acid-adsorptive porous membrane, a porous membrane (film thickness: 80 μm) prepared by subjecting a porous membrane of triacetylcellulose to a saponifying treatment was used and received in a receiving part of nucleic acid-adsorptive porous membrane of the cartridge for separation of nucleic acid which was prepared in the above (1).

(3) Preparation of RNA Solubilizing Reagent and Washing Solution

An RNA solubilizing reagent and a washing solution were prepared according to the following formulations.

| | | |
|---|---|---|
| (Formulations for RNA solubilizing reagent) | | |
| Guanidine hydrochloride (manufactured by Life | 382 | g |
| Technology) | | |
| Tris (manufactured by Life Technology) | 12.1 | g |
| Triton X-100 (manufactured by ICN) | 10 | g |
| Distilled water | 1,000 | ml |
| (Formulation for washing solution) | | |
| 10 mmol/L Tris-HCl (pH 7.5) 30% ethanol | | |
| (Formulation for recovering solution) | | |
| 1 mmol/L Tris-HCl (pH 6.5) | | |

(4) Purifying Operation for Nucleic Acid

Incubated solution of cancerous human bone cells (HL 60) was prepared. The incubated solution was collected so as to make cell numbers 1×10⁶, the cells were precipitated by centrifugation at 300×5 g and the supernatant liquid was removed to give the cells. To the above HL 60 cells (1×10⁶) was added 200 μl of the above RNA solubilizing reagent solution followed by stirring, 200 μl of ethanol was added thereto and the mixture was stirred to give a sample solution containing RNA. The sample solution containing RNA was infused into one of the openings of a nucleic acid purifying unit having porous membrane of an organic macromolecular substance comprising a mixture of acetylcelluloses having different acetyl values prepared in the above (1) and (2), then an apparatus for generation of pressure difference is connected to said one of the openings, inner part of the unit for separation and purification of nucleic acid is made in a compressed state and the infused sample solution containing RNA was passed through the above porous membrane so as to contact to the porous membrane and then discharged from another opening of the unit for separation and purification of nucleic acid. After that, a washing solution was infused into said one of the openings of the above unit for separation and purification of nucleic acid, an apparatus for generation of pressure difference is connected to said one of the openings, inner part of the unit for separation and purification of nucleic acid is made in a compressed state and the infused washing solution was passed through the above porous membrane and discharged from another opening. Then, a recovering solution was infused into said one of the openings of the above unit for separation and purification of nucleic acid, an apparatus for generation of pressure difference is connected to said one of the openings of the unit for separation and purification of nucleic acid, inner area of the cartridge for separation and purification of nucleic acid was made into a pressurized state, the infused recovering solution was passed through the porous membrane and discharged from another opening and the solution was recovered.

(5) Confirmation of Separation and Purification of RNA

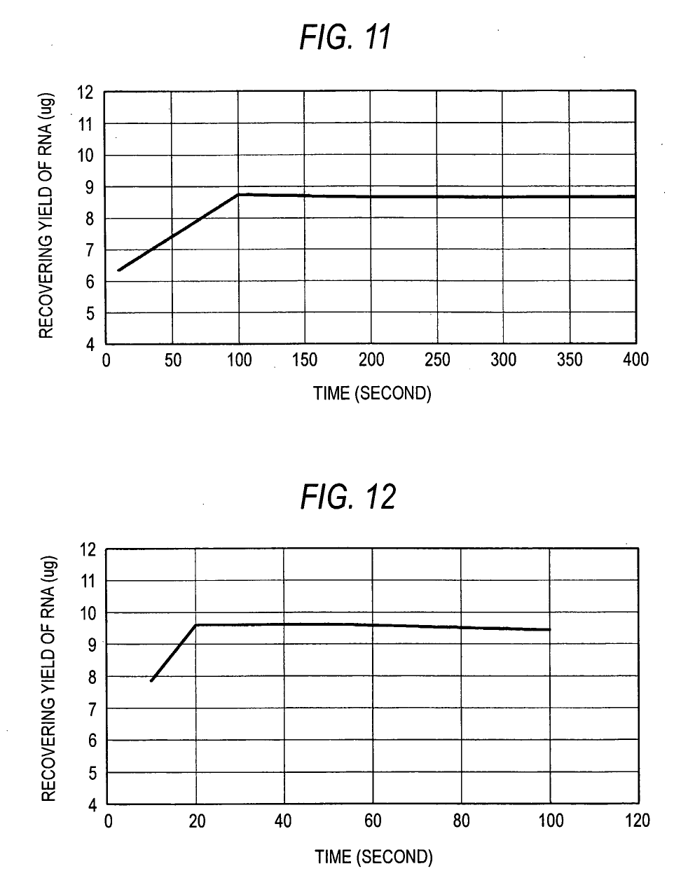

Absorption spectrum of the recovering solution at 260 nm was measured to determine the yield of RNA and evaluation of the yield of RNA to the time for being allowed to stand of the washing solution and recovering solution in the nucleic acid extracting step of this Example was conducted. As a result of this Example, the relation between the yield of nucleic acid and the time for being allowed to stand after infusion of the washing solution was shown in FIG. 11. Further, the relation between the yield of nucleic acid and the time for being allowed to stand after infusion of the recovering solution was shown in FIG. 12.

It has now been found that, as shown in FIG. 11 and FIG. 12, yield of RNA is significantly improved when the time for being allowed to stand after infusion of the washing solution and the time for being allowed to stand after infusion of the recovering solution are controlled so as to make them predetermined time. To be more specific, it has been found that, as shown in FIG. 11, yield of RNA is significantly improved when the infused washing solution is allowed to stand for 50 seconds or longer and then allowed to stand with RNA. It has been also found that, as shown in FIG. 12, yield of RNA is significantly improved when the infused recovering solution is allowed to stand for 20 seconds or longer and then allowed to stand with RNA.

### INDUSTRIAL APPLICABILITY

According to the present invention, in a method of separating and purifying nucleic acid by adsorption of nucleic acid in a sample solution containing nucleic acid with a nucleic acid-adsorptive porous membrane followed by subjecting to desorption via washing, etc., it is now possible, in a compact and less expensive manner, to constitute a method for extracting nucleic acid and also an apparatus for extracting nucleic acid where a sample solution containing nucleic acid is able to be prepared in good efficiency, simplicity, quickness, good automation adaptability and good reproducibility.

The entire disclosure of each and every foreign patent application from which the benefit of foreign priority has been claimed in the present application is incorporated herein by reference, as if fully set forth.

1. A method for extracting nucleic acid comprising:
adsorbing nucleic acid to a nucleic acid-adsorptive solid carrier by contacting a sample solution containing nucleic acid with the nucleic acid-adsorptive solid carrier;
washing the nucleic acid-adsorptive solid carrier by contacting a washing solution with the nucleic acid-adsorptive solid carrier, while the nucleic acid is adsorbed to the nucleic acid-adsorptive solid carrier; and
desorbing the nucleic acid from the nucleic acid-adsorptive solid carrier by contacting a recovering solution with the nucleic acid-adsorptive solid carrier,
wherein at least one of a time where the washing solution is dispensed and allowed to stand; and a time where the recovering solution is dispensed and allowed to stand is controlled to a predetermined time.

2. The method according to claim 1,
wherein the time where the washing solution is allowed to stand is controlled to a range of 50 seconds to 1,000 seconds.

3. The method according to claim 1,
wherein the time where the washing solution is allowed to stand is controlled to a range of 100 seconds to 300 seconds.

4. The method according to claim 1,
wherein the time where the recovering solution is allowed to stand is controlled to a range of 20 seconds to 300 seconds.

5. The method according to claim 1,
wherein the time where the recovering solution is allowed to stand is controlled to a range of 25 seconds to 60 seconds.

6. The method according to claim 1,
wherein the nucleic acid-adsorptive solid carrier is a nucleic acid-adsorptive solid carrier that adsorbs nucleic acid by an interaction involving substantially no ionic bond.

7. The method according to claim 1,
wherein a method for preparing the sample solution containing nucleic acid comprises:
mixing a pretreating solution containing a compound selected from a chaotropic salt, a surfactant, an antifoaming agent, a proteinase and a nucleic acid stabilizer with a test sample, so as to obtain a mixed solution; and
adding a water-soluble organic solvent to the mixed solution.

8. The method according to claim 7,
wherein the nucleic acid stabilizer is a mercapto compound.

9. The method according to claim 7,
wherein the chaotropic salt is a guanidium salt.

10. The method according to claim 7,
wherein the water-soluble organic solvent comprises at least one of methanol, ethanol, propanol and butanol.

11. The method according to claim 1,
wherein the nucleic acid-adsorptive solid carrier is received in an inner part of a container having at least two openings of a nucleic acid-extracting cartridge, and
wherein the method comprises:
adsorbing the nucleic acid in the sample solution to the nucleic acid-adsorptive solid carrier by a pressure difference after dispensing the sample solution containing the nucleic acid to the nucleic acid-extracting cartridge;
removing impurities by a pressure difference after dispensing the washing solution to the nucleic acid-extracting cartridge; and
separating the nucleic acid adsorbed to the nucleic acid-adsorptive solid carrier from the nucleic acid-adsorptive solid carrier by a pressure difference after dispensing the recovering solution to the nucleic acid-extracting cartridge, so as to recover the nucleic acid along with the recovering solution.

12. A nucleic acid-extracting apparatus for conducting a method according to claim 11,
wherein a nucleic acid-adsorptive solid carrier is a filter material.

13. The nucleic acid-extracting apparatus according to claim 12,
wherein the nucleic acid-extracting apparatus automatically carries out an extracting operation by a pressurization, and
wherein the extracting operation comprises:
adsorbing a nucleic acid in a sample solution to the filter material after dispensing the sample solution containing the nucleic acid to a nucleic acid-extracting cartridge;
removing impurities after dispensing a washing solution to the nucleic acid-extracting cartridge; and
separating the nucleic acid adsorbed to the filter material from the filter material after dispensing a recovering solution to the nucleic acid-extracting cartridge, so as to recover the nucleic acid along with the recovering solution.

14. The nucleic acid-extracting apparatus according to claim 12, comprising:
a compressed air supplying mechanism that introduces a compressed air from a pressurizing nozzle to the nucleic acid-extracting cartridge; and
a dispensing mechanism comprising a first dispensing nozzle that dispenses a washing solution to the nucleic acid-extracting cartridge and a second dispensing nozzle that dispenses a recovering solution to the nucleic acid-extracting cartridge.

15. The nucleic acid-extracting apparatus according to claim 12, comprising:
a retaining mechanism that retains a plurality of arranged nucleic acid-extracting cartridges and a plurality of arranged recovering containers for receiving the recovering solution containing nucleic acid;
a compressed air supplying mechanism that introduces a compressed air from a single pressurizing nozzle to the plurality of arranged nucleic acid-extracting cartridges;
a dispensing mechanism comprising a first dispensing nozzle that dispenses a washing solution to the plurality of arranged nucleic acid-extracting cartridges and a second dispensing nozzle that dispenses a recovering solution to the plurality of arranged nucleic acid-extracting cartridges; and
a transfer means that relatively transfers one of the single pressurizing nozzle of the compressed air supplying mechanism and the retaining mechanism against the other.

16. The nucleic acid-extracting apparatus according to claim 12,
wherein the plurality of arranged nucleic acid-extracting cartridges are supported at a fixed side, and
wherein the single pressurizing nozzle of the compressed air supplying mechanism is supported in movable in an arranging direction of the plurality of arranged nucleic acid-extracting cartridges.

17. The nucleic acid-extracting apparatus according to claim 12,
wherein at least one of the first dispensing nozzle and the second dispensing nozzle, and the single pressurizing nozzle are installed in an unified transferable matter.

18. The nucleic acid-extracting apparatus according to claim 12,
wherein the single pressurizing nozzle of the compressed air supplying mechanism is supported at a fixed side, and
wherein the plurality of arranged nucleic acid-extracting cartridges are supported in movable in an arranging direction of the plurality of arranged nucleic acid-extracting cartridges.

19. The nucleic acid-extracting apparatus according to claim 12,
wherein the plurality of nucleic acid-extracting cartridges are installed in a same arranging distance, and
wherein an arranging distance from each of the first dispensing nozzle and the second dispensing nozzle to the single pressurizing nozzle is an integral multiple of the same arranging distance of the plurality of nucleic acid-extracting cartridges.
